(19) United States
(12) Patent Application Publication
(10) Pub. No.: US 2009/0202986 A1
(43) Pub. Date: Aug. 13, 2009

(54) METHODS OF OPTIMIZING ANTIBODY VARIABLE REGION BINDING AFFINITY

(76) Inventors: William D. Huse, Del Mar, CA (US); Jeffry Dean Watkins, Encinitas, CA (US); Alain Philippe Vasserot, Carlsbad, CA (US); David Matthew Marquis, Encinitas, CA (US); Eric Michael Smith, San Diego, CA (US)

Correspondence Address: ELI LILLY & COMPANY, PATENT DIVISION, P.O. BOX 6288, INDIANAPOLIS, IN, 46206-6288, US

(21) Appl. No.: 11/577,061

(22) PCT Filed: Oct. 28, 2004
(86) PCT No.: PCT/US04/32772

§ 371 Date: Dec. 15, 2008

Related U.S. Application Data
(60) Provisional application No. 60/620,694, filed on Oct. 22, 2004.

(51) Int. Cl. C12Q 1/68

(52) U.S. Cl. 435/6

(57) ABSTRACT

The present invention provides optimized heteromeric variable region binding fragments and antibodies comprising optimized heteromeric variable region binding fragments. Preferably, the optimized heteromeric variable region binding fragments exhibit optimized activity compared to donor heteromeric variable regions and have unvaried human frameworks. The present invention also provides methods of making the optimized heteromeric variable region binding fragments.

### FIELD OF THE INVENTION

The present invention provides optimized heteromeric variable region binding fragments and antibodies comprising optimized heteromeric variable region binding fragments. Preferably, the optimized heteromeric variable region binding fragments exhibit optimized activity compared to donor heteromeric variable regions and have unvaried human frameworks. The present invention also provides methods of making the optimized heteromeric variable region binding fragments.

### BACKGROUND OF THE INVENTION

The war on cancer is entering its third decade and recent years have shown tremendous progress in the understanding of cancer development and progression yet there has been only marginal decreases in death rates from most types of cancer. Standard chemotherapy and radiation therapy generally involve treatment with therapeutic agents that impact not only cancer cells but other highly proliferative cells of the body, often leading to debilitating side effects. Thus, it is desirable to identify therapeutic agents with a higher degree of specificity for the carcinogenic lesion.

The discovery of monoclonal antibodies (mabs) in the 1970's provided great hope for the reality of creating therapeutic molecules with high specificity. Antibodies that bind to tumor antigens would provide specific targeting agents for cancer therapy. However, while the development of monoclonal antibodies has provided a valuable diagnostic reagent, certain limitations restrict their use as therapeutic entities.

A limitation encountered when attempts are made to use mAbs as therapeutic agents is that since mAbs are developed in non-human species, usually mouse, they elicit an immune response in human patients. Chimeric antibodies join the variable region of the non-human species, which confers binding activity, to a human constant region. However, the chimeric antibody is often still immunogenic and it is therefore necessary to further modify the variable region.

One modification is the grafting of complementarity-determining regions, (CDRs) which impart antigen binding onto a human antibody variable framework. However, this approach is imperfect because CDR grafting often diminishes the binding activity of the resulting humanized mAb. Attempts to regain binding activity require laborious, step-wise procedures which have been pursued essentially by a trial and error type of approach. For example, one difficulty in regaining binding affinity is because it is difficult to predict which framework residues serve a critical role in maintaining antigen binding affinity and specificity. Consequently, while antibody humanization methods that rely on structural and homology data are used, the complexity that arises from the large number of framework residues potentially involved in binding activity has prevented success.

Combinatorial methods have been applied to restore binding affinity, however, these methods require sequential rounds of mutagenesis and affinity selection that can both be laborious and unpredictable.

Thus, there exists a need for efficient and reliable methods for producing human monoclonal antibodies which exhibit comparable or enhanced binding affinities to their non-human counterparts. The present invention satisfies this need and provides related advantages as well.

### SUMMARY OF THE INVENTION

The present invention provides optimized heteromeric variable region binding fragments and antibodies comprising optimized heteromeric variable region binding fragments. Preferably, the optimized heteromeric variable region binding fragments exhibit optimized activity compared to donor heteromeric variable regions and have unvaried human frameworks. The present invention also provides methods of making the optimized heteromeric variable region binding fragments.

In some embodiments, the present invention provides an optimized heteromeric variable region (e.g. that may or may not be part of a full antibody other molecule) having an optimized activity such as higher antigen binding affinity than a donor heteromeric variable region, wherein the donor heteromeric variable region comprises three light chain donor CDRs, and wherein the optimized heteromeric variable region comprises: a) a light chain altered variable region comprising; i) four unvaried (e.g., unvaried human, bovine, canine, feline, porcine, etc.) germline light chain framework regions, and ii) three light chain altered variable region CDRs, wherein at least one of the three light chain altered variable region CDRs is a light chain donor CDR variant, and wherein the light chain donor CDR variant comprises a different amino acid at one, two, three or four positions compared to one of the three light chain donor CDRs (e.g. the at least one light chain donor CDR variant is identical to one of the light chain donor CDRs except for one, two, three or four amino acid differences). In further embodiments, the optimized heteromeric variable region comprises a heavy chain variable region. In certain embodiments, three of the four unvaried human germline light chain framework regions are from a human kappa light chain gene selected from the group consisting of: A11, A17, A18, A19, A20, A27, A30, L1, L11, L12, L2, L5, L6, L8, O12, O2, and O8.

In other embodiments, the present invention provides an optimized heteromeric variable region (e.g. that may or may not be part of a full antibody other molecule) having an optimized activity, such as higher antigen binding affinity than a donor heteromeric variable region, wherein the donor heteromeric variable region comprises three heavy chain donor CDRs, and wherein the optimized heteromeric variable region comprises: a) a heavy chain altered variable region comprising; i) four unvaried (e.g., unvaried human, bovine, canine, feline, porcine, etc.) germline heavy chain framework regions, and ii) three heavy chain altered variable region CDRs, wherein at least one of the three heavy chain altered variable region CDRs is a heavy chain donor CDR variant, and wherein the heavy chain donor CDR variant comprises a different amino acid at one, two, three or four positions compared to one of the three heavy chain donor CDRs (e.g. the at least one heavy chain donor CDR variant is identical to one of the heavy chain donor CDRs except for one, two, three or four amino acid differences). In further embodiments, the optimized heteromeric variable region comprises a light chain variable region. In other embodiments, three of the four unvaried human germline heavy chain framework regions are from a human heavy chain gene selected from the group consisting of: VH2-5, VH2-26, VH2-70, VH3-20, VH3-72, VH1-46, VH3-9, VH3-66, VH3-74, VH4-31, VH1-18, VH1-69, VH3-7, VH3-11, VH3-15, VH3-21, VH3-23, VH3-30, VH3-48, VH4-39, VH4-59, and VH5-51.

In additional embodiments, the present invention provides an optimized heteromeric variable region (e.g. that may or may not be part of a full antibody molecule) having an optimized activity such as higher antigen binding affinity than a donor heteromeric variable region, wherein the donor heteromeric variable region comprises three light chain donor CDRs and three heavy chain donor CDRs, and wherein the optimized heteromeric variable region comprises: a) a light chain altered variable region comprising; i) four unvaried (e.g., unvaried human, bovine, canine, feline, porcine, etc.) germline light chain framework regions, and ii) three light chain altered variable region CDRs, wherein at least one of the three light chain altered variable region CDRs is a light chain donor CDR variant, and wherein the light chain donor CDR variant comprises a different amino acid at one, two, three or four positions compared to one of the three light chain donor CDRs, and b) a heavy chain altered variable region comprising; i) four unvaried (e.g., unvaried human, bovine, canine, feline, porcine, etc.) germline heavy chain framework regions, and ii) three heavy chain altered variable region CDRs, wherein at least one of the three heavy chain altered variable region CDRs is a heavy chain donor CDR variant, and wherein the heavy chain donor CDR variant comprises a different amino acid at only one, two, three, or four positions compared to one of the three heavy chain donor CDRs.

In certain embodiments, three of the four unvaried germline light chain framework regions (e.g. FRL1, FRL2, and FRL3) are from a human kappa light chain gene selected from the group consisting of: A11, A17, A18, A19, A20, A27, A30, L1, L11, L12, L2, L5, L6, L8, O12, O2, and O8. In other embodiments, three of the four unvaried human germline heavy chain framework regions (e.g. FRH1, FRH2, and FRH3) are from a human heavy chain gene selected from the group consisting of: VH2-5, VH2-26, VH2-70, VH3-20, VH3-72, VH1-46, VH3-9, VH3-66, VH3-74, VH4-31, VH1-18, VH1-69, VH3-7, VH3-11, VH3-15, VH3-21, VH3-23, VH3-30, VH3-48, VH4-39, VH4-59, and VH5-51. In some embodiments, the optimized heteromeric variable region binding fragment is capable of binding von Willebrand Factor (vWF) or other proteins involved with human disease.

In additional embodiments, the present invention provides an antibody comprising an optimized heteromeric variable region with optimized activity such as having higher antigen binding affinity than a donor heteromeric variable region, wherein the donor heteromeric variable region comprises three light chain donor CDRs and three heavy chain donor CDRs, and wherein the optimized heteromeric variable region comprises: a) a light chain altered variable region comprising; i) four unvaried (e.g., unvaried human, bovine, canine, feline, porcine, etc.) germline light chain framework regions, and ii) three light chain altered variable region CDRs, wherein at least one of the three light chain altered variable region CDRs is a light chain donor CDR variant, and wherein the light chain donor CDR variant comprises a different amino acid at one, two, three or four positions compared to one of the three light chain donor CDRs, and b) a heavy chain altered variable region comprising; i) four unvaried (e.g., unvaried human, bovine, canine, feline, porcine, etc.) germline heavy chain framework regions, and ii) three heavy chain altered variable region CDRs, wherein at least one of the three heavy chain altered variable region CDRs is a heavy chain donor CDR variant, and wherein the heavy chain donor CDR variant comprises a different amino acid at one, two, three, or four positions compared to one of the three heavy chain donor CDRs. In some embodiments, the antibody further comprises a C_{L} region and a C₁ region. In other embodiments, the antibody further comprises an Fc region. In certain embodiments, the antibody further comprises a second heteromeric variable region binding fragment (e.g. an optimized heteromeric variable region binding fragment). In certain embodiments, the donor heteromeric variable region is non-human (e.g. rat, mouse, monkey, etc.) and the unvaried light and heavy chain frameworks are human.

In some embodiments, the present invention provides methods of expressing a heteromeric variable region (which may or may not be part of a larger molecule such an antibody) with optimized activity such as having higher antigen binding affinity than a donor heteromeric variable region, wherein the donor heteromeric variable region comprises three light chain donor CDRs and three heavy chain donor CDRs, and wherein the method comprises; a) providing; i) a first oligonucleotide encoding an altered light chain variable region, wherein the altered light chain variable region comprises: A) four unvaried human germline light chain framework regions, wherein three of the four unvaried human germline light chain framework regions are from a human kappa light chain gene selected from the group consisting of: A11, A17, A18, A19, A20, A27, A30, L1, L11, L12, L2, L5, L6, L8, O12, O2, and O8; and B) three light chain altered variable region CDRs, wherein at least one of the three light chain altered variable region CDRs is a light chain donor CDR variant, and wherein the light chain donor CDR variant comprises a different amino acid at one, two, three or four positions compared to one of the three light chain donor CDRs, and ii) a second oligonucleotide encoding an altered heavy chain variable region, wherein the altered heavy chain variable region comprises; A) four unvaried human germline heavy chain framework regions, wherein three of the four unvaried human germline heavy chain framework regions are from a human heavy chain gene selected from the group consisting of: VH2-5, VH2-26, VH2-70, VH3-20, VH3-72, VH1-46, VH3-9, VH3-66, VH3-74, VH4-31, VH1-18, VH1-69, VH3-7, VH3-11, VH3-15, VH3-21, VH3-23, VH3-30, VH3-48, VH4-39, VH4-59, and VH5-51; and B) three heavy chain altered variable region CDRs, wherein at least one of the three heavy chain altered variable region CDRs is a heavy chain donor CDR variant, and wherein the heavy chain donor CDR variant comprises a different amino acid at one, two, three, or four positions compared to one of the heavy chain donor CDRs, and b) expressing the first and second oligonucleotides under conditions such that a heteromeric variable region binding fragment is generated that exhibits higher antigen binding affinity than the donor heteromeric variable region.

In additional embodiments, the present invention provides methods of expressing a heteromeric variable region having higher antigen binding affinity (or other optimized activity) than a donor heteromeric variable region, wherein the donor heteromeric variable region comprises three light chain donor CDRs and three heavy chain donor CDRs, the method comprising; a) providing; i) first oligonucleotides encoding four unvaried human germline light chain framework regions, wherein three of the four unvaried human germline light chain framework regions are from a human kappa light chain gene selected from the group consisting of: A11, A17, A18, A19, A20, A27, A30, L1, L11, L12, L2, L5, L6, L8, O12, O2, and O8; ii) a population of second oligonucleotides encoding: A) first light chain CDRs, wherein the first light chain CDRs comprise donor CDR variants, wherein the donor CDR variants comprise a different amino acid at only one, two, three or four positions compared to one of the three light chain donor CDRs, B) second light chain CDRs, wherein the second light chain CDRs encode each of the three light chain donor CDRs; iii) third oligonucleotides encoding four unvaried human germline heavy chain framework regions, wherein three of the four unvaried human germline heavy chain framework regions are from a human heavy chain gene selected from the group consisting of: VH2-5, VH2-26, VH2-70, VH3-20, VH3-72, VH1-46, VH3-9, VH3-66, VH3-74, VH4-31, VH1-18, VH1-69, VH3-7, VH3-11, VH3-15, VH3-21, VH3-23, VH3-30, VH3-48, VH4-39, VH4-59, and VH5-51; and iv) a population of fourth oligonucleotides encoding: A) first heavy chain CDRs, wherein the first heavy chain CDRs comprise donor CDR variants, wherein the donor CDR variants comprise a different amino acid at one, two, three or four positions compared to one of the three heavy chain donor CDRs, and B) second heavy chain CDRs, wherein the second heavy chain CDRs encode each of the three heavy chain donor CDRs; b) mixing the first oligonucleotides and the population of second oligoncucleotides such that a population of fifth oligonucleotides encoding light chain variable regions is generated, wherein at least one of the light chain variable regions encoded by the population of fifth oligonucleotides comprises i) an unvaried human germline light chain framework, and ii) at least one light chain donor CDR variant; c) mixing the third oligonucleotides and the population of fourth oligonucleotides such that a population of sixth oligonucleotides encoding heavy chain variable regions is generated, wherein at least one of the heavy chain variable regions encoded by the population of sixth oligonucleotides comprises; i) an unvaried human germline heavy chain framework, and ii) at least one heavy chain donor CDR variant; and d) expressing the fifth and sixth populations of oligonucleotides to produce combinations of heteromeric variable region binding fragments. In other embodiments, the method further comprises step e) identifying at least one heteromeric variable region having higher antigen binding affinity than the donor heteromeric variable region.

In some embodiments, the unvaried human germline light chain framework regions comprises FR1, FR2, FR3 and FR4 regions configured to hybridize to the light chain donor CDRs and the light chain donor CDR variants such that the population of fifth oligonucleotides encoding light chain variable regions is generated. In other embodiments, the unvaried human germline heavy chain framework regions comprise FR1, FR2, FR3 and FR4 regions configured to hybridize to the heavy chain donor CDRs and the heavy chain donor CDR variants such that the population of fifth oligonucleotides encoding heavy chain variable regions is generated.

In other embodiments, one of the three light chain altered variable region CDRs is identical to one of the three light chain donor CDRs. In some embodiments, two of the three light chain altered variable region CDRs are each identical to one of the three light chain donor CDRs. In certain embodiments, one of the three heavy chain altered variable region CDRs is identical to one of the three heavy chain donor CDRs. In additional embodiments, two of the three heavy chain altered variable region CDRs are each identical to one of the three light chain donor CDRs. In certain embodiments, at least two of the three light chain altered variable region CDRs are light chain donor CDR variants. In other embodiments, three of the three light chain altered variable region CDRs are light chain donor CDR variants. In some embodiments, at least two of the three heavy chain altered variable region CDRs are heavy chain donor CDR variants. In yet other embodiments, three of the three heavy chain altered variable region CDRs are heavy chain donor CDR variants. In certain embodiments, the donor heteromeric variable region is murine.

In other embodiments, the higher antigen binding affinity is at least 2-fold higher antigen binding affinity (e.g. about 2-3 fold higher or about 2-20 fold higher). In particular embodiments, the higher antigen binding affinity is 3-fold higher antigen binding affinity. In certain embodiments, the higher antigen binding affinity is at least: 5-fold higher, 8-fold higher, or 10-fold higher. In other embodiments, the higher antigen binding affinity is at least 12-fold, 15-fold, 20-fold, 25-fold, 50-fold, 100-fold, or 1000-fold higher (e.g. 10-50 fold or 20-500 fold higher).

In some embodiments, the optimized activity is an increased association rate (kₒₙ) for an antigen (e.g. 2-fold to 50-fold increased association rate compared to the donor). In certain embodiments, the optimized activity is a decreased disassociation rate (k_{off}) for an antigen (e.g. a 2-fold, 50-fold, 100-fold, 500-fold or 100-fold decrease in disassociation rate compared to the donor). In particular embodiments, the optimized activity is selected from higher binding affinity, increased/decreased association rate, or increased/decreased disassociation rate—all as compared to the donor. In other embodiments, the optimized activity is an increased or decreased catalytic rate, disassociation constant or association constant (e.g. for a catalytic heteromeric variable region) as compared to the donor heteromeric variable region.

In some embodiments, the donor CDRs and the altered variable region CDRs are as defined by Chothia. In other embodiments, the donor CDRs and the altered variable region CDRs are as defined by Kabat. In particular embodiments, the donor CDRs and the altered variable region CDRs are as defined by MacCallum. In preferred embodiments, the donor CDRs and the altered variable region CDRs are as defined by the combined definitions of Kabat and Chothia. In other preferred embodiments, the donor and altered variable region CDRs are as defined by Kabat, except CDRH1 is defined by the combined definition of Kabat and Chothia.

In certain embodiments, three of the four unvaried human germline light chain framework regions are LFR1. LFR2, and LFR3 all of which are from the same human germline light chain variable region gene. In other embodiments, three of the four unvaried human germline heavy chain framework regions are HFR1, HFR2, and HFR3 all of which are from the same human germline heavy chain variable region gene.

In certain embodiments, the present invention provides heteromeric variable region binding fragments and antibodies that are able to bind to human von Willebrand factor (vWF) and comprise an unvaried human framework. In particular embodiments, the unvaried human framework is a human germline framework.

In some embodiments, the present invention provides compositions comprising a vWF binding molecule (or nucleic acid sequence encoding a vWF binding molecule), wherein the vWF binding molecule comprises: a) a light chain variable region, or a portion of a light chain variable region, wherein the light chain variable region (or the portion) comprises; i) a CDRL1 amino acid sequence as shown in SEQ ID NO:9. In certain embodiments, the vWF binding molecule has a CDRL2 amino acid sequence selected from SEQ ID NO:11, 13, 15, 17, 19, and 21. In further embodiments, the vWF binding molecule has a CDRL3 amino acid sequence as shown in SEQ ID NO:23.

In certain embodiments, the present invention provides compositions comprising a heavy chain variable region (or portion thereof), or a nucleic acid sequence (or portion thereof) encoding a heavy chain variable region, wherein the heavy chain variable region (or portion thereof) comprises: a) a CDRH1 amino acid sequence as shown in SEQ ID NO:25. In other embodiments, the heavy chain variable region comprises a CDRH2a amino acid sequence selected from the group consisting of SEQ ID NO:27, 29, and 31. In some embodiments, the heavy chain variable region comprises a CDRH2b amino acid sequence selected from the group consisting of SEQ ID NO:33, 35, 37, 39, and 41. In further embodiments, the heavy chain variable region comprises a CDRH3 amino acid sequence selected from the group consisting of SEQ ID NO:43, 45, 47, and 49.

In some embodiments, the present invention provides compositions comprising a nucleic acid molecule encoding a light chain variable region of a vWF binding molecule, wherein the nucleic acid molecule comprises a CDRL1 nucleic acid sequence as shown in SEQ ID NO:10 or a nucleic acid sequence encoding the same peptide as SEQ ID NO:10 (e.g., due to the degeneracy of the genetic code, many nucleic acid sequences may be constructed that code for the same peptide as encoded by SEQ ID NO:10; this same principle is true for any sequences described herein. In certain embodiments, the vWF binding molecule has a CDRL2 nucleic acid sequence selected from SEQ ID NO:12, 14, 16, 18, 20, and 22, or a nucleic acid sequence encoding the same peptides as SEQ ID NO:12, 14, 16, 18, 20, or 22. In further embodiments, the vWF binding molecule has a CDRL3 amino acid sequence as shown in SEQ ID NO:24, or a nucleic acid sequence encoding the same peptide as SEQ ID NO:24.

In certain embodiments, the present invention provides compositions comprising a nucleic acid sequence (or portion thereof) encoding a heavy chain variable region, wherein the heavy chain variable region (or portion thereof) comprises: a) a CDRH1 nucleic acid sequence as shown in SEQ ID NO:26. In other embodiments, the present invention provides a nucleic acid sequence encoding a CDRH2a as shown in SEQ ID NO:28, 30, or 32. In some embodiments, the present invention provides a nucleic acid sequence encoding a CDRH2b as shown in SEQ ID NO:34, 36, 38, 40, or 42. In further embodiments, the present invention provides a CDRH3 as shown in SEQ ID NO:44, 46, 48, or 50.

In some embodiments, the present invention provides a peptide comprising at least two (or at least three or four) CDRs selected from the group consisting of SEQ ID NO:9, 11, 13, 15, 17, 19, 21, 23, 25, 27, 29, 31, 33, 35, 37, 39, 41, 43, 45, 47,and49. In certain embodiments, the light and/or heavy chain variable region comprises a portion of a framework (e.g. containing 2 or 3 subregions, such as FR2 and FR3). In some embodiments, the light and/or heavy chain variable region comprises a fully human framework. In other embodiments, the light and/or heavy chain variable region comprise a human germline framework.

In some embodiments, the vWF binding molecule comprises an antibody or antibody fragment (e.g., Fv, Fab, etc.). In particular embodiments, the vWF binding molecule comprises the C1 Fv or Fab. In other embodiments, the vWF binding molecule comprises the C4 Fv or Fab. In additional embodiments, the vWF binding molecule comprises an Fv or Fab selected from the group consisting of C7, C8, C9, and C4-4.

In certain embodiments, the vWF binding molecule is contained within a host cell (e.g. eukaryotic, or prokaryotic host cell). In other embodiments, the nucleic acid sequence encoding the light and/or heavy chain is contained within a plasmid or other expression vector.

In some embodiments, the present invention provides methods of inhibiting the binding of vWF to the GPIb protein comprising: a) providing; i) a subject, and ii) a composition, wherein the composition comprises a vWF binding molecule of the present invention (e.g. acting as an anti-thrombotic agent); and b) administering the composition to the subject. In certain embodiments, the administering inhibits RIPA (ristocetin-induced platelet aggregation), BIPA (botrocetin-induced platelet aggregation), or SIPA (shear stress-induced platelet aggregation)—type reactions in human patients. In other embodiments, the present invention provides methods of treating a disease comprising: a) providing; i) a subject symptoms of the disease, and ii) a composition, wherein the composition comprises the vWF binding molecules of the present invention; and b) administering the composition to the subject such that the symptoms are reduced and/or being eliminated. In particular embodiments, the vWF binding molecules are applied to prevent or treat diseases relevant to platelet adhesion and aggregation, including, but not limited to, the treatment of transient cerebral ischemic attack, unstable angina pectoris, cerebral infarction, myocardial infarction, and peripheral arterial occlusive disease, prevention of reocclusion after PTCA and occlusion of coronary artery by-pass graft, and for the treatment of coronary artery valve replacement and essential thrombocythemia.

In some embodiments, amino acid modification(s) are introduced into the CH2 domain of an Fc region of a vWF binding molecule or any of the binding molecules (e.g. antibodies) disclosed herein. Useful amino acid positions for modification to generate a variant IgG Fc region with altered Fc gamma receptor (FcγR) binding affinity or activity include any one or more of the following amino acid positions: 268, 269, 270, 272, 276, 278, 280, 283, 285, 286, 289, 290, 292, 293, 294, 295, 296, 298, 300 301, 303, 305, 307, 309, 331, 332, 333, 334, 335, 337, 338, 340, 360, 373, 376, 416, 419, 430, 434, 435, 437, 438 or 439 of the Fc region of a binding molecule of the present invention. In preferred embodiments, the parent Fc region used as the template to generate such variants comprises a human IgG Fc region. In some embodiments, to generate an Fc region variant with reduced binding to the FcγR one may introduce an amino acid modification at any one or more of the following amino acid positions: 252, 254, 265, 268, 269, 270, 272, 278, 289, 292, 293, 294, 295, 296, 298, 300, 301, 303, 322, 324, 327, 329, 332, 333, 335, 338, 340, 373, 376, 382, 388, 389, 414, 416, 419, 434, 435, 437, 438 or 439 of the Fc region of a vWF, or other type of, binding molecule. In particular embodiments, Fc region variants with improved binding to one or more FcγRs may also be made. Such Fc region variants may comprise an amino acid modification at any one or more of the following amino acid positions: 280, 283, 285, 286, 290, 294, 295, 298, 300, 301, 305, 307, 309, 312, 315, 331, 332, 333, 334, 337, 340, 360, 378, 398 or 430 of the Fc region of a binding molecule as disclosed herein. In certain embodiments, the amino acid modification is Y3001, which may also be combined with any other suggested modification described herein.

In other embodiments, the amino acid modification is Y300L. In some embodiments, the amino acid modification is Q295K or Q295L. In certain embodiments, the amino acid modification is E294N. In other embodiments, the amino acid modification at position 296 is Y296P. In some embodiments, the amino acid modification at position 298 is S298P. In other embodiments, the amino acid modification is S289N, S298P, S298V or S298D (any of these particular modifications may also be combined with any other modification described herein.) In certain embodiments, the vWF binding molecule, or other type of binding molecules (e.g. heteromeric variable regions) comprises a heavy chain constant region mutation. In other embodiments, the binding molecule comprises a heavy chain constant region with a mutation selected from D280H and K290S. Additional Fc mutations or changes in Fe glycosylation are described in U.S. Pat. No. 6,528,624; WO0042072; U.S. Pat. No. 5,648,260; U.S. Pat. No. 6,180,377; WO0179299; WO9958572; WO9823289; WO8907142; U.S. Pat. No. 5,834,597; U.S. Pat. No. 6,602,684; WO9858964; and WO9730087; all of which are incorporated herein by reference.

Alternatively or additionally, it may be useful to combine amino acid modifications with one or more further amino acid modifications that alter C1q binding and/or the complement dependent cytotoxicity function of the Fc region of a binding molecule as disclosed herein. The starting polypeptide of particular interest may be one that binds to C1q and displays complement dependent cytotoxicity (CDC). Amino acid substitutions described herein may serve to alter the ability of the starting polypeptide to bind to C1q and/or modify its complement dependent cytotoxicity function (e.g., to reduce and preferably abolish these effector functions). However, polypeptides comprising substitutions at one or more of the described positions with improved C1q binding and/or complement dependent cytotoxicity (CDC) function are also contemplated herein. For example, the starting polypeptide may be unable to bind C1q and/or mediate CDC and may be modified according to the teachings herein such that it acquires these further effector functions. Moreover, polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

One type of amino acid substitution serves to alter the glycosylation pattern of the Fc region of a binding molecule. This may be achieved, for example, by deleting one or more glycosylation site(s) found in the polypeptide, and/or adding one or more glycosylation sites that are not present in the polypeptide. Glycosylation of an Fc region is typically either N-linked or O-linked. N-linked refers to the attachment of the carbohydrate moiety to the side chain of an asparagine residue. The peptide sequences asparagine-X-serine and asparagine-X-threonine, where X is any amino acid except proline, are the recognition sequences for enzymatic attachment of the carbohydrate moiety to the asparagine side chain. Thus, the presence of either of these peptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the Fc region of a binding molecule (e.g. heteromeric variable region) is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites).

In certain embodiments, the binding molecules of the present invention are expressed in cells that express beta(1,4)-N-acetylglucosaminyltransferase III (GnT III), such that GnT III adds GlcNAc to the binding molecules. Methods for producing binding molecules in such a fashion are provided in WO9954342, WO03011878, patent publication 20030003097A1, and Umana et al., Nature Biotechnology, 17:176-180, February 1999; all of which are herein specifically incorporated by reference in their entireties.

In certain embodiments, the present invention provides kits comprising: a) a binding molecule of the present invention (e.g. heteromeric variable region); and b) instructions for using the binding molecule to treat a disease in a subject or instructions for employing the binding molecule for scientific research or diagnostic purposes (e.g., for performing ELISA assays, etc.). In some embodiments, the present invention provides cell lines stably or transiently transfected with nucleic acid sequences encoding the binding molecules of the present invention.

In some embodiments, the present invention provides methods of conferring donor CDR binding affinity onto an antibody acceptor variable region framework, comprising: (a) constructing a population of altered antibody variable region encoding nucleic acids, the population comprising encoding nucleic acids for an acceptor variable region framework containing a plurality of different amino acids at one or more acceptor framework region amino acid positions and donor CDRs containing a plurality of different amino acids at one or more donor CDR amino acid positions; (b) expressing the population of altered variable region encoding nucleic acids, and (c) identifying one or more altered variable regions having binding affinity substantially the same or greater than the donor CDR variable region.

In certain embodiments, the one or more altered variable regions are identified by comparing the relative binding of the altered variable regions to the donor CDR variable region. In other embodiments, the one or more altered variable regions are identified by measuring the binding affinity of the altered variable regions. In particular embodiments, the one or more altered variable regions are identified by measuring the association rate (kon) or disassociation rate (koff) of the altered variable regions. In additional embodiments, the acceptor variable region framework is a heavy chain variable region framework. In other embodiments, the acceptor variable region framework is a light chain variable region framework. In some embodiments, the framework amino acid positions are located in framework region 1, framework region 2, framework region 3 or framework region 4. In particular embodiments, the donor CDR amino acid positions is located in CDR1, CDR2, or CDR3.

In particular embodiments, the one or more amino acid positions in the acceptor framework region is selected by differences in amino acid identity between corresponding positions in donor and acceptor framework regions. In further embodiments, the one or more amino acid positions in the acceptor framework region is selected as being a canonical framework residue. In other embodiments, the one or more amino acid positions in the acceptor framework region is selected as being exposed to solvent. In some embodiments, the one or more amino acid positions in the acceptor framework region is selected by a characteristic within the group consisting of being proximal to a CDR, predicted to contact the opposite domain in the VL-VH interface, lack of relatedness to the donor framework amino acid position at that position, and predicted to modulate CDR activity.

In certain embodiments, the one or more amino acid positions in the donor CDR is selected as being a CDR residue as defined by Kabat. In some embodiments, the altered variable regions are coexpressed with a light chain variable region. In additional embodiments, the altered variable region is coexpressed with a heavy chain variable region.

In some embodiments, the present invention provides methods of simultaneously grafting and optimizing the binding affinity of a variable region binding fragment, comprising: (a) constructing a population of altered heavy chain variable region encoding nucleic acids comprising an acceptor variable region framework containing donor CDRs and a plurality of different amino acids at one or more framework region and CDR amino acid positions; (b) constructing a population of altered light chain variable region encoding nucleic acids comprising an acceptor variable region framework containing donor CDRs and a plurality of different amino acids at one or more framework regions and CDR amino acid positions; (c) coexpressing the populations of heavy and light chain variable region encoding nucleic acids to produce diverse combinations of heteromeric variable region binding fragments, and (d) identifying one or more heteromeric variable region binding fragments having affinity substantially the same or greater than the donor CDR heteromeric variable region binding fragment.

In certain embodiments, the one or more heteromeric variable region binding fragments are identified by comparing the relative binding of the heteromeric variable region binding fragments to the donor CDR heteromeric variable region binding fragment. In some embodiments, the one or more heteromeric variable region binding fragments are identified by measuring the binding affinity of the heteromeric variable region binding fragments. In particular embodiments, the one or more heteromeric variable region binding fragments are identified by measuring the association rate (kon) or disassociation rate (koff) of the heteromeric variable region binding fragments. In further embodiments, the framework amino acid positions are located in framework region 1, framework region 2, framework region 3 or framework region 4. In other embodiments, the donor CDR amino acid positions is located in CDR1, CDR2, or CDR3.

In certain embodiments, the one or more amino acid positions in the acceptor framework region is selected by differences in amino acid identity between corresponding positions in donor and acceptor framework regions. In other embodiments, the one or more amino acid positions in the acceptor framework region is selected as being a canonical framework residue. In particular embodiments, the one or more amino acid positions in the acceptor framework region is selected as being exposed to solvent. In other embodiments, the one or more amino acid positions in the acceptor framework region is selected by a characteristic within the group consisting of being proximal to a CDR, predicted to contact the opposite domain in the VL-VH interface, lack of relatedness to the donor framework amino acid position at that position, and/or predicted to modulate CDR activity. In some embodiments, the one or more amino acid positions in the donor CDR is selected as being a CDR residue as defined by Kabat.

In particular embodiments, the present invention provides methods of optimizing the binding affinity of an antibody variable region, comprising: (a) constructing a population of antibody variable region encoding nucleic acids from a parent variable region encoding nucleic acid, the population comprising two or more CDRs containing a plurality of different amino acids at one or more CDR amino acid positions; (b) expressing the population of variable region encoding nucleic acids, and (c) identifying one or more variable regions having binding affinity substantially the same or greater than the parent variable region.

In some embodiments, the one or more variable regions are identified by comparing the relative binding of the variable regions to the parent variable region. In other embodiments, the one or more variable regions are identified by measuring the binding affinity of the variable regions. In certain embodiments, the one or more variable regions are identified by measuring the association rate (kon) or disassociation rate (koff) of the variable regions. In other embodiments, the variable region is a heavy chain variable region. In some embodiments, the variable region is a light chain variable region. In some embodiments they are combinations of the above.

In particular embodiments, the two or more CDRs are selected from the group consisting of CDR1, CDR2, or CDR3. In some embodiments, the one or more amino acid positions in the two or more CDRs is selected as being a CDR residue as defined by Kabat. In other embodiments, the variable regions are coexpressed with a light chain variable region. In certain embodiments, the variable regions are coexpressed with a heavy chain variable region. In particular embodiments, the antibody variable region is selected from the group consisting of native, grafted, altered, and optimized variable regions.

In some embodiments, the present invention provides methods of optimizing the activity of a catalytic antibody variable region, comprising: (a) constructing a population of heavy chain variable region encoding nucleic acids from a parent heavy chain variable region encoding nucleic acid, the population comprising two or more CDRs containing a plurality of different amino acids at one or more CDR amino acid positions; (b) constructing a population of light chain variable region encoding nucleic acids from a parent light chain variable region encoding nucleic acid, the population comprising two or more CDRs containing a plurality of different amino acids at one or more CDR amino acid positions; (c) coexpressing the population of heavy and light chain variable region encoding nucleic acids containing the two or more CDRs having the plurality of different amino acids at one or more CDR positions to produce diverse combinations of heteromeric variable region catalytic fragments, and (d) identifying one or more heteromeric variable regions having optimized catalytic activity compared to the parent catalytic antibody variable region.

In other embodiments, the one or more heteromeric variable regions are identified by comparing the relative catalytic activity of the heteromeric variable regions to the parent variable region. In some embodiments, the one or more heteromeric variable regions are identified by measuring a substrate association rate (kon), a substrate disassociation rate (koff), substrate binding affinity, a transition state binding affinity, a turnover rate or a Km. In particular embodiments, the two or more CDRs are selected from the group consisting of CDR1, CDR2, and CDR3. In certain embodiments, the one or more amino acid positions in the two or more CDRs is selected as being a CDR residue as defined by Kabat.

The invention provides a method of conferring donor CDR binding affinity onto an antibody acceptor variable region framework. The method consists of: (a) constructing a population of altered antibody variable region encoding nucleic acids, said population comprising encoding nucleic acids for an acceptor variable region framework containing a plurality of different amino acids at one or more acceptor framework region amino acid positions and donor CDRs containing a plurality of different amino acids at one or more donor CDR amino acid positions; (b) expressing said population of altered variable region encoding nucleic acids, and (c) identifying one or more altered variable regions having binding affinity substantially the same or greater than the donor CDR variable region. The acceptor variable region framework can be a heavy or light chain variable region framework and the populations of heavy and light chain altered variable regions can be expressed alone to identify heavy or light chains having binding affinity substantially the same or greater than the donor CDR variable region. The populations of heavy and light chains additionally can be coexpressed to identify heteromeric altered variable region binding fragments. The invention also provides a method of simultaneously grafting and optimizing the binding affinity of a variable region binding fragment. The method consists of: (a) constructing a population of altered heavy chain variable region encoding nucleic acids comprising an acceptor variable region framework containing donor CDRs and a plurality of different amino acids at one or more framework region and CDR amino acid positions; (b) constructing a population of altered light chain variable region encoding nucleic acids comprising an acceptor variable region framework containing donor CDRs and a plurality of different amino acids at one or more framework regions and CDR amino acid positions; (c) coexpressing said populations of heavy and light chain variable region encoding nucleic acids to produce diverse combinations of heteromeric variable region binding fragments, and (d) identifying one or more heteromeric variable region binding fragments having affinity substantially the same or greater than the donor CDR heteromeric variable region binding fragment. A method of optimizing the binding affinity of an antibody variable region is also provided. The method consists of: (a) constructing a population of antibody variable region encoding nucleic acids, said population comprising two or more CDRs containing a plurality of different amino acids at one or more CDR amino acid positions; (b) expressing said population of variable region encoding nucleic acids, and (c) identifying one or more variable regions having binding affinity substantially the same or greater than the donor CDR variable region. The variable region populations can be heavy or light chains and can be expressed as individual populations or they can be coexpressed to produce heteromeric variable region binding fragments.

### BRIEF DESCRIPTION OF THE DRAWINGS

FIG. 1 (SEQ ID NO:s 1-4) shows the alignment of anti-CD40 variable region and human template amino acid sequences.

FIG. 2 shows binding reactivity of humanized anti-CD40 variants.

FIG. 3 shows molecular modeling of anti-CD40 variant CW43.

FIG. 4 shows a comparison of the quantitation of murine framework residues in active variants from two libraries.

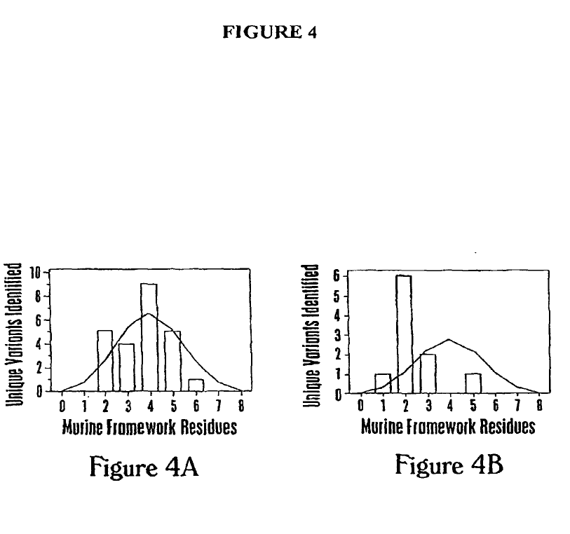

FIG. 5 shows an alignment of NMC-4 with human germline sequences. Vertical lines denote differences in sequences. The work of Kabat was used to number residues and define CDR's (underlined) with the exception of CDR-H1 with used Kabat and Chothia residues combined.

FIG. 6 shows the results of a vWF ELISA with the combinatorial clones described in Example 2.

### DEFINITIONS

To facilitate an understanding of the invention, a number of terms are defined below.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat(1) | Chothia(2) | MacCallum(3) |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

(1)Residue numbering follows the nomenclature of Kabat et al., supra
(2)Residue numbering follows the nomenclature of Chothia et al., supra
(3)Residue numbering follows the nomenclature of MacCallum et al., supra

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

As used herein, the term “framework region” is intended to mean each domain of the framework that is separated by the CDRs. Therefore, for the specific example of a heavy chain variable region and for the CDRs as defined by Kabat et al., framework region 1 corresponds to the domain of the variable region encompassing amino acids 1-30; region 2 corresponds to the domain of the variable region encompassing amino acids 36-49; region 3 corresponds to the domain of the variable region encompassing amino acids 66-94, and region 4 corresponds to the domain of the variable region from amino acids 103 to the end of the variable region. The framework regions for the light chain are similarly separated by each of the light claim variable region CDRs. Similarly, using the definition of CDRs by Chothia et al. or McCallum et al. the framework region boundaries are separated by the respective CDR termini as described above.

As used herein, the term “donor” is intended to mean a parent antibody molecule or fragment thereof from which a portion is derived from, given or contributes to another antibody molecule or fragment thereof so as to confer either a structural or functional characteristic of the parent molecule onto the receiving molecule. For the specific example of CDR grafting, the parent molecule from which the grafted CDRs are derived is a donor molecule. The donor CDRs confer binding affinity of the parent molecule onto the receiving molecule. It should be understood that a donor molecule does not have to be from a different species as the receiving molecule of fragment thereof. Instead, it is sufficient that the donor is a separate and distinct molecule.

As used herein, the term “acceptor” is intended to mean an antibody molecule or fragment thereof which is to receive the donated portion from the parent or donor antibody molecule or fragment thereof. An acceptor antibody molecule or fragment thereof is therefore imparted with the structural or functional characteristic of the donated portion of the parent molecule. For the specific example of CDR grafting, the receiving molecule for which the CDRs are grafted is an acceptor molecule. The acceptor antibody molecule or fragment is imparted with the binding affinity of the donor CDRs or parent molecule. As with a donor molecule, it is understood that an acceptor molecule does not have to be from a different species as the donor.

A “variable region” when used in reference to an antibody or a heavy or light chain thereof is intended to mean the amino terminal portion of an antibody which confers antigen binding onto the molecule and which is not the constant region. The term is intended to include functional fragments thereof which maintain some of all of the binding function of the whole variable region. Therefore, the term “heteromeric variable region binding fragments” is intended to mean at least one heavy chain variable region and at least one light chain variable regions or functional fragments thereof assembled into a heteromeric complex. Heteromeric variable region binding fragments include, for example, functional fragments such as Fab, F(ab)2, Fv, single chain Fv (scFv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990).

As used herein, the term “population” is intended to refer to a group of two or more different molecules. A population can be as large as the number of individual molecules currently available to the user or able to be made by one skilled in the art. Populations can be as small as 2-4 molecules or as large as 10¹³ molecules. An example where a small population can be useful is where one wishes to optimize binding affinity of a variable region or of heteromeric binding fragments by compiling beneficial differences from a small number of parent molecules having similar binding affinity into a single variable binding fragment species. An example of where large populations, including as large as 10⁸ or greater different molecules, can be desired is where all possible combinations of amino acids differences between donor and acceptor at all positions within a variable region are to be generated to obtain maximum diversity and increase the efficiency of compiling beneficial changes. In some embodiments, populations are between about 5 and 10 different species as well as up to hundreds or thousands of different species. The populations can be diverse or redundant depending on the intent and needs of the user. Those skilled in the art will know what size and diversity of a population is suitable for a particular application.

As used herein, the term “altered” when used in reference to an antibody variable region is intended to mean a heavy or light chain variable region that contains one or more amino acid changes in a framework region, a CDR or both compared to the parent amino acid sequence at the changed position. Where an altered variable region is derived from or composed of different donor and acceptor regions, the changed amino acid residues within the altered species are to be compared to their respective amino acid positions within the parent donor and acceptor regions. For example, a variable region containing donor CDRs grafted into an acceptor framework and containing one or more amino acid changes within the framework regions and one or more amino acid changes within the CDRs will have amino acids residues at the changed framework region positions different than the residues at the comparable positions in the acceptor framework. Similarly, such an altered variable region will have amino acid residues at the changed CDR positions different than the residues at the comparable positions in the donor CDRs.

As used herein, the term “nucleic acid” or “nucleic acids” is intended to mean a single- or double-stranded DNA or RNA molecule. A nucleic acid molecule of the invention can be of linear, circular, or branched configuration, and can represent either the sense or antisense strand, or both, of a nucleic acid molecule. The term also is intended to include nucleic acid molecules of both synthetic and natural origin. A nucleic acid molecule of natural origin can be derived from any animal, such as a human, non-human primate, mouse, rat, rabbit, bovine, porcine, ovine, canine, feline, or amphibian, or from a lower eukaryote, such as Drosophila, C. elegans or yeast. A synthetic nucleic acid includes, for example, chemical and enzymatic synthesis. The term “nucleic acid” or “nucleic acids” is similarly intended to include analogues of natural nucleotides which have similar functional properties as the referenced nucleic acid and which can be utilized in a manner similar to naturally occurring nucleotides and nucleosides.

As used herein, the term “coexpressing” is intended to mean the expression of two or more molecules by the same cell. The coexpressed molecules can be polypeptides or encoding nucleic acids. The coexpression can be, for example, constitutive or inducible. Such nucleic acid sequences can also be expressed simultaneously or, alternatively, regulated independently. Various combinations of these modes of coexpression can additionally be used depending on the number and intended use of the variable region encoding nucleic acids. The term is intended to include the coexpression of members originating from different populations in the same cell. For example, populations of molecules can be coexpressed where single or multiple different species from two or more populations are expressed in the same cell. A specific example includes the coexpression of heavy and light chain variable region populations where at least one member from each population is expressed together in the same cell to produce a library of cells coexpressing different species of heteromers variable region binding fragments. Populations which can be coexpressed can be as small as 2 different species within each population. Additionally, the number of molecules coexpressed from different populations also can be as large as 10⁸ or greater, such as in the case where multiple amino acid position changes of multiple framework regions or CDRs in both heavy and light chain antibody variable region populations are produced and coexpressed. Numerous different sized populations of encoding nucleic acids in between the above ranges and greater can also be coexpressed. Those skilled in the art know, or can determine, what modes of coexpression can be used to achieve a particular goal or satisfy a desired need.

As used herein, the term “identifying” is intended to mean detecting by a qualitative or quantitative means, a variable region or altered variable region of the invention by functional or biochemical properties, including, for example, binding affinity of catalytic activity.

As used herein the term “binding affinity” is intended to mean the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent heteromeric variable region binding fragment can exhibit altered or optimized binding affinity due to its valency.

As used herein, the term “optimizing” when used in reference to a variable region or a functional fragment thereof is intended to mean that the binding affinity of the variable region has been modified compared to the binding affinity of a parent variable region or a donor variable region. A variable region exhibiting optimized activity can exhibit, for example, higher affinity or lower affinity binding, or increased or decreased association or dissociation rates compared to an unaltered variable region. A variable region exhibiting optimized activity also can exhibit increased stability such as increased half-life in a particular organism. For example, an antibody activity can be optimized to increase stability by decreasing susceptibility to proteolysis. An antibody exhibiting optimized activity also can exhibit lower affinity binding, including decreased association rates or increased dissociation rates, if desired. An optimized variable region exhibiting lower affinity binding is useful, for example, for penetrating a solid tumor. In contrast to a higher affinity variable region, which would bind to the peripheral regions of the tumor but would be unable to penetrate to the inner regions of the tumor due to its high affinity, a lower affinity variable region would be advantageous for penetrating the inner regions of the tumor. As with optimization of binding affinities above, optimization of a catalytic variable region can be, for example, increased or decreased catalytic rates, disassociation constants or association constants.

As used herein, the term “substantially the same” when used in reference to binding affinity is intended to mean similar or identical binding affinities where one molecule has a binding affinity constant that is similar to another molecule within the experimental variability of the affinity measurement. The experimental variability of the binding affinity measurement is dependent upon the specific assay used and is known to those skilled in the art.

### DETAILED DESCRIPTION OF THE INVENTION

The invention is directed to a method of conferring donor CDR binding affinity onto an antibody acceptor variable region framework. The method effectively combines CDR grafting procedures and affinity reacquisition of the grafted variable region into a single step. The methods of the invention also are applicable for affinity maturation of an antibody variable region. The affinity maturation process can be substituted for, or combined with the affinity reacquisition function when being performed during a CDR grafting procedure. Alternatively, the affinity maturation procedure can be performed independently from CDR grafting procedures to optimize the binding affinity of a variable region, or an antibody. An advantage of combining grafting and affinity reacquisition procedures, or affinity maturation, is the avoidance of time consuming, step-wise procedures to generate a grafted variable region, or antibody, which retains sufficient binding affinity for therapeutic utility. Therefore, therapeutic antibodies can be generated rapidly and efficiently using the methods of the invention. Such advantages beneficially increase the availability and choice of useful therapeutics for human diseases as well as decrease the cost to the developer and ultimately to the consumer.

In one embodiment, the invention is directed to methods of producing grafted heavy and light chain variable regions having similar or better binding affinity as the CDR donor variable region. When coexpressed, the grafted heavy and light chain variable regions assemble into variable region binding fragments having similar or better binding affinity as the donor antibody or variable region binding fragments thereof. The grafting is accomplished by generating a diverse library of CDR grafted variable region fragments and then screening the library for binding activity similar or better than the binding activity of the donor. A diverse library is generated by selecting acceptor framework positions that differ at the corresponding position compared to the donor framework and making a library population containing all possible amino acid residue changes at each of those positions together with all possible amino acid residue changes at each position within the CDRs of the variable region. The grafting is accomplished by splicing a population of encoding nucleic acids for the donor CDR containing species representing all possible amino acid residues at each CDR position into a population of encoding nucleic acids for an antibody acceptor variable region framework which contains species representing all possible amino acid residue changes at the selected framework positions. The resultant population encodes the authentic donor and acceptor framework amino acid sequences as well as all possible combinations and permutations of these sequences with each, for example, of the 20 naturally occurring amino acids at the changed positions.

In another embodiment, the invention is directed to methods of producing grafted heavy and light chain variable regions, and heteromeric binding fragments thereof, having similar or better binding affinity as the CDR donor variable region. As described above, the grafting is accomplished by generating a diverse library of CDR grafted variable region fragments and then screening the library for binding activity similar or better than the binding activity of the donor. However, the diverse library is generated by selecting acceptor framework positions that are predicted to affect CDR binding affinity and making a library population containing all possible amino acid residue changes at each of those positions or subsets of the selected amino acid positions together with all possible amino acid residue changes at each position within the CDRs of the variable region, or subsets of CDR positions. The grafting is accomplished by splicing a population of encoding nucleic acids for the donor CDR containing the selected position changes into a population of encoding nucleic acids for an antibody acceptor variable region framework which contains the selected position changes.

In yet another embodiment, the invention is directed to the optimization of binding affinity of an antibody variable region. The optimization is accomplished by generating a library of variable regions containing all possible amino acid residue changes at each amino acid position within two or more CDRs. When expressed and screened for binding activity, the variable region, or heavy and light chain heteromeric binding fragments, those species within the population are selected that contain increased or decreased binding activity compared to the parent molecule as optimal binders. Libraries containing subsets, representing less than all amino acid positions within the CDRs, can be generated similarly and screened for selecting optimal binding variable regions and heteromeric binding fragments thereof.

The invention provides a method for conferring donor CDR binding affinity onto an antibody acceptor variable region framework. The method consists of: (a) constructing a population of altered antibody variable region encoding nucleic acids, the population consisting of encoding nucleic acids for an acceptor variable region framework containing a plurality of different amino acids at one or more acceptor framework region amino acid positions and donor CDRs containing a plurality of different amino acids at one or more donor CDR amino acid positions; (b) expressing the population of altered variable region encoding nucleic acids, and (c) identifying one or more altered variable regions having binding affinity substantially the same or greater than the donor CDR variable region.

The process of producing human antibody forms from nonhuman species involves recombinantly splicing CDRs from a nonhuman donor antibody into a human acceptor framework region to confer binding activity onto the resultant grafted antibody, or variable region binding fragment thereof. The process of grafting, referred to as the procedure for splicing CDRs into a framework, while mechanically simple it almost always results in a grafted antibody that exhibits a substantial loss in binding affinity. Although donor and acceptor variable regions are structurally similar, the process nevertheless combines CDR binding domains with a heterologous acceptor region, resulting in a conformationally imperfect setting for the binding residues of the grafted antibody. Therefore, once the CDR-grafted antibody, or variable region binding fragment is made, it requires subsequent rounds of molecular engineering to reacquire binding affinity comparable to the donor antibody. The present invention combines these steps such that CDR grafting and binding reacquisition occur in a single simultaneous procedure. The method is also applicable to optimizing the binding affinity of an antibody, or variable region binding fragment simultaneous with CDR grafting and to optimizing an antibody or variable region binding fragment in a single procedure without including the CDR grafting process.

The methods of the invention confer or impart donor CDR binding affinity onto an antibody acceptor variable region framework in a procedure which achieves grafting of donor CDRs and affinity reacquisition in a simultaneous process. The methods similarly can be used, either alone or in combination with CDR grafting, to modify or optimize the binding affinity of a variable region. The methods for conferring donor CDR binding affinity onto an acceptor variable region are applicable to both heavy and light chain variable regions and as such can be used to simultaneous graft and optimize the binding affinity of an antibody variable region.

The methods for conferring donor CDR binding affinity onto a variable region involve identifying the relevant amino acid positions in the acceptor framework that are known or predicted to influence a CDR conformation, or that are known or predicted to influence the spacial context of amino acid side chains within the CDR that participate in binding, and then generate a population of altered variable region species that incorporate a plurality of different amino acid residues at those positions. For example, the different amino acid residues at those positions can be incorporated either randomly or with a predetermined bias and can include all of the twenty naturally occurring amino acid residues at each of the relevant positions. Subsets, including less than all of the naturally occurring amino acids can additionally be chosen for incorporation at the relevant framework positions. Including a plurality of different amino acid residues at each of the relevant framework positions ensures that there will be at least one species within the population that will have framework changes which allows the CDRs to reacquire their donor binding affinity in the context of the acceptor framework variable region.

In addition to the framework changes at selected amino acid positions, the CDRs also are altered to contain a plurality of different amino acid residue changes at all or selected positions within the donor CDRs. For example, random or biased incorporation of the twenty naturally occurring amino acid residues, or preselected subsets, are also introduced into the donor CDRs to produce a diverse population of CDR species. Including a diverse population of different CDR variant species ensures that beneficial changes in the framework positions are not neutralized by a conformationally incompatible residue in a donor CDR. Inclusion of CDR variant species into the diverse population of variable regions also allows for the generation of variant species that exhibit optimized binding affinity for a predetermined antigen.

The resultant population of CDR grafted variable regions described above will therefore contain, at the relevant framework positions and at the selected CDR positions, a species corresponding to the authentic parent amino acid residue at each position as well as a diverse number of different species which correspond to the possible combinations and permutations of the authentic parent amino acid residues together with the variant residues at each of the relevant framework and selected CDR positions. Such a diverse population of CDR grafted variable regions are screened for an altered variable region species which retains donor CDR binding activity, or optimized binding activity.

One advantage of the methods of the invention is that they do not limit the choice of acceptor variable regions applicable, or expected to be successful, for receiving CDRs from the donor molecule. For example, when choosing an acceptor region it can be desirable, or in some circumstances even required, to select an acceptor that is closely similar to the variable region amino acid sequence harboring the donor CDRs because the CDR conformation in the grafted variable region will likely be more similar to that of the donor. However, selecting similar framework region sequences between the donor and acceptor variable regions still does not provide which residues, out of the differences, actually play a role in CDR binding affinity of the grafted variable region. Selection of similar acceptor frameworks therefore only limits the number of possible residues which to investigate to reacquire binding affinity onto the grafted variable region. The methods of the invention circumvent this problem by producing a library of all possible or relevant changes in the acceptor framework, and then screening those variable regions, or heteromeric binding fragments thereof for species that maintain or exhibit increased binging affinity compared to the donor molecule. Therefore, the applicability is not preconditioned on the availability or search for an acceptor framework variable region similar to that of the donor.

Selection of the relevant framework amino acid positions to alter can depend on a variety of criteria well known to those skilled it the art. As described above, one criteria for selecting relevant framework amino acids to change can be the relative differences in amino acid framework residues between the donor and acceptor molecules. Selection of relevant framework positions to alter using this approach is simple and has the advantage of avoiding any subjective bias in residue determination or any inherent bias in CDR binding affinity contribution by the residue. Criteria other than relatedness of amino acid residues can be used for selecting relevant framework positions to alter. Such criteria can be used in combination with, or alternative to the selection of framework positions having divergent amino acid residues. These additional criteria are described further and similarly are well known to those skilled in the art.

Another criteria which can be used for determining the relevant amino acid positions to change can be, for example, selection of framework residues that are known to be important, or contribute to CDR conformation. For example, canonical framework residues play such a role in CDR conformation or structure. Such residues can be considered to be relevant to change for a variety of reasons, including for example, their new context of being associated with heterologous CDR sequences in the grafted variable region. Targeting of a canonical framework residue as a relevant position to change can identify a more compatible amino acid residue in context with its associated donor CDR sequence. Additionally, targeting of canonical residues can allow for the identification of residues at these positions that absorb detrimental effects to CDR structure from residues located elsewhere in the framework region.

The frequency of an amino acid residue at a particular framework position is another criteria which can be used for selecting relevant framework amino acid positions to change. For example, comparison of the selected framework with other framework sequences within its subfamily can reveal residues that occur at minor frequencies at a particular position or positions. Such positions harboring less abundant residues are similarly applicable for selection as a position to alter in the acceptor variable region framework.

The relevant amino acid positions to change also can be selected, for example, based on proximity to a CDR. In certain contexts, such residues can participate in CDR conformation or antigen binding. Moreover, this criteria can similarly be used to prioritize relevant positions selected by other criteria described herein. Therefore, differentiating between residues proximal and distal to one or more CDRs is an efficient way to reduce the number of relevant positions to change using the methods of the invention.

Other criteria for selecting relevant amino acid framework positions to alter include, for example, residues that are known or predicted to reside in three-dimensional space near the antigen-CDR interface or predicted to modulate CDR activity. Similarly, framework residues that are known or predicted to contact opposite domain of the heavy (VH) and light (VL) chain variable region interface. Such framework positions can effect the conformation or affinity of a CDR by modulating the CDR binding pocket, antigen interaction or the VH and VL interaction. Therefore, selection of these amino acid positions as relevant for construction of the diverse population to screen can beneficially identify framework changes which replace residues having detrimental effects on CDR conformation or absorb detrimental effects of residues occurring elsewhere in the framework.

Finally, other framework residues that can be selected for alteration include amino acid positions that are inaccessible to solvent. Such residues are generally buried in the variable region and therefore capable of influencing the conformation of the CDR or VH and VL interactions. Solvent accessibility can be predicted, for example, from the relative hydrophobicity of the environment created by the amino acid side chains of the polypeptide or by known three-dimensional structural data.

In addition to selecting the relevant framework positions, the method of conferring donor CDR binding affinity onto an antibody acceptor variable region framework also incorporates changes in the donor CDR amino acid positions. As with selecting the relevant framework positions to change, there is similarly a range of possible changes that can be made in the donor CDR positions. Some or all of the possible changes that can be selected for change can be introduced into the population of grafted donor CDRs to practice the methods of the invention.

One approach is to change all amino acid positions along a CDR by replacement at each position with, for example, natural or synthetically produced any amino acid. The replacement of each position can occur in the context of other donor CDR amino acid positions so that a significant portion of the CDR maintains the authentic donor CDR sequence, and therefore, the binding affinity of the donor CDR. For example, an acceptor variable region framework targeted for relevant amino acid positions changes as described previously, can be targeted for grafting with a population of CDRs containing single position replacements at each position within the CDRs. Similarly, an acceptor variable region framework can be targeted for grafting with a population of CDRs containing more than one position changed to incorporate all twenty amino acid residues, or a fractional subset, at each set of positions within the CDRs. For example, all possible sets of changes corresponding to two, three or four or more amino acid positions within a CDR can be targeted for introduction into a population of CDRs for grafting into an acceptor variable region framework.

Single amino acid position changes are generated at each position without altering the remaining amino acid positions within the CDR. A population of single position changes will contain at each position the varied amino acid residues, incorporated either randomly or with a biased frequency, while leaving the remaining positions as donor CDR residues. For the specific example of a ten residue CDR, the population will contain species having the first, second, and third, continued through the tenth CDR residue, individually randomized and/or represented by a biased frequency of incorporated amino acid residues while the remaining non-varied positions represent the donor CDR amino acid residues. For the specific example described above, these non-varied positions would correspond to positions 2-10; 1,3-10; 1,2,4-10, continued through positions 1-9, respectively. Therefore, the resultant population will contain species that represent all single position changes.

Similarly, double, triple, and quadruple amino acid position changes can be generated for each set of positions without altering the remain amino acid positions within the CDR. For example, a population of double position changes will contain at each set of two positions the varied amino acid residues while leaving the remaining positions as donor CDR residues. The sets will correspond to, for example, positions I and 2, 1 and 3, 1 and 4, etc., through the set corresponding to the first and last position of the CDR. The population will also contain sets corresponding to positions 2 and 3, 2 and 4, 2 and 5, etc., through the set corresponding to the second an last position of the CDR. Likewise, the population will contain sets of double position changes corresponding to all pairs of position changes beginning with position three of the CDR. Similar pairs of position changes are made with the remaining sets CDR amino acid positions. Therefore, the population will contain species that represent every pairwise combination of amino acid position changes. In a similar fashion, populations corresponding to sets of changes representing all triple and/or quadruplet changes along a CDR can similarly be targeted for grafting into the variable region frameworks using the methods of the invention.

The above populations of CDR variant species can be targeted for any or all of the CDRs which constitute the binding pocket of a variable region. Therefore, an acceptor variable region framework targeted for relevant amino acid positions changes as described previously, can be targeted for the simultaneous incorporation of donor CDR variant populations at one, two, or all three recipient CDR locations. The choice of which CDR or the number of CDRs to target with amino acid position changes will depend on, for example, whether a full CDR grafting into an acceptor is desired or whether the method is being performed for optimization of binding affinity. Many grafting procedures will generally employ the grafting of all three CDRs, where at least one of the CDRs will contain amino acid positions changes. Generally however, all of the donor CDRs will be populations containing amino acid position changes. Converesly, and as described further below, optimization procedures can employ CDR variant populations corresponding to any, up to and including all of the CDRs within a variable region.

Another approach for selecting donor CDR amino acids to change for conferring donor CDR binding affinity onto an antibody acceptor variable region framework is to select known or readily identifiable CDR positions that are highly variable. For example, the variable region CDR 3 is generally highly variable due to genetic recombination. This region therefore can be selectively targeted for amino acid position changes during grafting procedures to ensure binding affinity reacquisition or augmentation when made together with relevant acceptor variable framework changes as described herein.

In contrast, CDR residues that appear conserved or that have been empirically determined to be non-mutable (e.g., by functional criteria) will generally be avoided when selecting residues in the CDR to target for change. It should be noted, however, that apparent non-mutable residues can nevertheless be successfully changed using the methods of the invention because the populations of altered variable regions contain from a few to many amino acid position changes in both the framework regions and in the CDR regions. As such, the CDR grafted variable regions identified by binding affinity are a result of the all the changes and therefore, all the interactions of residues introduced into a particular species. Therefore, suboptimal residues incorporated at, for example, an apparent non-mutable position can be counteracted and even augmented by amino acid substitutions elsewhere in the framework regions or in other CDRs.

Similarly, because the methods of the invention for CDR grafting, affinity reacquisition and affinity optimization employ the production and screening of diverse populations of variable region species generated from an acceptor framework and donor CDR variants, there are numerous effects on binding affinity that will occur due to the combined interactions of two or more amino acid changes within a single variable region species. For example, the affect of amino acid changes in either a framework region or CDR that are inherently beneficial can be masked or neutralized due to surrounding authentic parent residues or due to their context in a heterologous region of a grafted antibody. However, second site changes in the surrounding residues or the heterologous regions can unveil the beneficial characteristics of the latent residue or residues. Such second site changes can occur, for example, in both proximal and distal heterologous or homologous region sequences.

For example, if the beneficial residue is in a grafted CDR region, the proximal heterologous sequences would be the adjacent framework regions whereas distal heterologous regions would be framework regions separated by an adjacent CDR. In this specific example, a proximal homologous region would be the surrounding residues within the grafted CDR harboring the beneficial change whereas the remaining CDRs are examples of distal homologous regions. By analogy, the opposite would be true for an inherently beneficial residue in a framework region. Specifically, proximal homologous region sequences would be located in the same framework region and distal homologous sequences would be in any of the other framework regions. Proximal heterologous region sequences would be in the adjacent CDR or CDRs whereas nonadjacent CDRs constitute distal heterologous region sequences. Such second site effects can occur, for example, through the translation of conformational changes to the CDR binding pocket or to the framework regions.

Other effects on binding affinity that can occur due to the combined interactions of two or more amino acid changes within a single variable region species include, for example, the neutralization or augmentation of inherently detrimental changes and the augmentation of beneficial amino acid changes or the augmentation of parent residues As with the unveiling of beneficial changes and the ability to counteract changes in apparently non-mutable residues, the neutralization and augmentation of amino acid changes within the grafted CDRs or framework region by second site changes can occur, for example, by imparting or translating conformational changes from the second site changes to the CDR binding pocket or to the framework regions. The second site changes can occur in any of the framework regions, including for example, framework regions 1 through 4 as well as in any of the three CDR regions. An advantage of the methods of the invention is that no prior information is required to assess which amino acid positions or changes can be inherently beneficial or detrimental, or which positions or changes can be further augmented by second site changes. Instead, by selecting relevant amino acid positions or subsets thereof in the acceptor variable region framework and CDRs, and generating a diverse population containing amino acid variants at these positions, combinations of beneficial changes occurring at the selected positions will be identified by screening for increased or optimized binding affinity of the CDR graft variable region. Such beneficial combinations can include the unveiling of inherently beneficial residues, neutralization of inherently detrimental residues and/or the augmentation of parent residues or functionally neutral changes.

Following selection of relevant amino acid positions in the framework regions and in the donor CDRs as described previously, amino acid changes at some or all of the selected positions are incorporated into encoding nucleic acids for the acceptor variable region framework and donor CDRs, respectively. Simultaneously with the incorporation of the encoding amino acid changes at the selected positions, the encoding nucleic acids sequences for each of the donor CDRs, including selected changes, are also incorporated into the acceptor variable region framework encoding nucleic acid to generate a population of altered variable region encoding nucleic acids.

An altered variable region of the invention will contain at least one framework position which variably incorporates different amino acid residues and at least one CDR position which variably incorporates different amino acid residues as described. The variability at any or all of the altered positions can range from a few to a plurality of different amino acid residues, including all twenty naturally occurring amino acids or functional equivalents and analogues thereof. The different species of the altered variable region containing the variable amino acid residues at one or more positions within the framework and CDR regions will make up the population from which to screen for an altered variable region having binding affinity substantially the same or greater than the donor CDR variable region.

Selection of the number and location of the amino acid positions to vary is flexible and can depend on the intended use and desired efficiency for identification of the altered variable region having substantially the same or greater binding affinity compared to the donor variable region. In this regard, the greater the number of changes that are incorporated into a altered variable region population, the more efficient it is to identify at least one species that exhibits substantially the same or greater binding affinity as the donor. Alternatively, where the user has empirical or actual data to the affect that certain amino acid residues or positions contribute disproportionally to binding affinity, then it can be desirable to produce a limited population of altered variable regions which focus on changes within or around those identified residues or positions.

For example, if CDR grafted variable regions are desired, a large, diverse population of altered variable regions can include all the non-identical framework region positions between the donor and acceptor framework and all single CDR amino acid position changes. Alternatively, a population of intermediate diversity can include, for example, subsets of only the proximal non-identical framework positions to be incorporated together with all single CDR amino acid position changes. The diversity of the above populations can be further increased, for example, by additionally including all pairwise CDR amino acid position changes. In contrast, populations focusing on predetermined residues or positions which incorporate variant residues at as few as one framework and one CDR amino acid position can be constructed similarly for screening and identification of an altered antibody variable region of the invention. As with the above populations, the diversity of such focused populations can be further increased by additionally expanding on the positions selected for change, for example, to include other relevant positions in either or both of the framework and CDR regions. There are numerous other combinations ranging from few changes to many changes in either or both of the framework regions and CDRs that can additionally be employed, all of which will result in a population of altered variable regions that can be screened for the identification of at least one CDR grafted altered variable region of the invention. Those skilled in the art will know, or can determine, which selected residue positions in the framework and/or donor CDRs, or subsets thereof, can be varied to produce a population for screening and identification of a altered antibody of the invention given the teachings and guidance provided herein.

Simultaneous incorporation of all of the CDR encoding nucleic acids and all of the selected amino acid position changes can be accomplished by a variety of methods known to those skilled in the art, including for example, recombinant and/or chemical synthesis. For example, simultaneous incorporation can be accomplished by, for example, chemically synthesizing the nucleotide sequence for the acceptor variable region, fused together with the donor CDR encoding nucleic acids, and incorporating at the positions selected for harboring variable amino acid residues a plurality of corresponding amino acid codons.

One such method well known in the art for rapidly and efficiently producing a large number of alterations in a known amino acid sequence or for generating a diverse population of variable or random sequences is known as codon-based synthesis or mutagenesis. This method is the subject matter of U.S. Pat. Nos. 5,264,563 and 5,523,388 and is also described in Glaser et al. J. Immunology 149:3903 (1992). Briefly, coupling reactions for the randomization of, for example, all twenty codons which specify the amino acids of the genetic code are performed in separate reaction vessels and randomization for a particular codon position occurs by mixing the products of each of the reaction vessels. Following mixing, the randomized reaction products corresponding to codons encoding an equal mixture of all twenty amino acids are then divided into separate reaction vessels for the synthesis of each randomized codon at the next position. For the synthesis of equal frequencies of all twenty amino acids, up to two codons can be synthesized in each reaction vessel.

Variations to these synthesis methods also exist and include, for example, the synthesis of predetermined codons at desired positions and the biased synthesis of a predetermined sequence at one or more codon positions. Biased synthesis involves the use of two reaction vessels where the predetermined or parent codon is synthesized in one vessel and the random codon sequence is synthesized in the second vessel. The second vessel can be divided into multiple reaction vessels such as that described above for the synthesis of codons specifying totally random amino acids at a particular position. Alternatively, a population of degenerate codons can be synthesized in the second reaction vessel such as through the coupling of NNG/T nucleotides where N is a mixture of all four nucleotides. Following the synthesis of the predetermined and random codons, the reaction products in each of the two reaction vessels are mixed and then redivided into an additional two vessels for synthesis at the next codon position.

A modification to the above-described codon-based synthesis for producing a diverse number of variant sequences can be employed similarly for the production of the variant populations described herein. This modification is based on the two vessel method described above which biases synthesis toward the parent sequence and allows the user to separate the variants into populations containing a specified number of codon positions that have random codon changes.

Briefly, this synthesis is performed by continuing to divide the reaction vessels after the synthesis of each codon position into two new vessels. After the division, the reaction products from each consecutive pair of reaction vessels, starting with the second vessel, is mixed. This mixing brings together the reaction products having the same number of codon positions with random changes. Synthesis proceeds then by dividing the products of the first and last vessel and the newly mixed products from each consecutive pair of reaction vessels and redividing into two new vessels. In one of the new vessels, the parent codon is synthesized and in the second vessel, the random codon is synthesized. For example, synthesis at the first codon position entails synthesis of the parent codon in one reaction vessel and synthesis of a random codon in the second reaction vessel. For synthesis at the second codon position, each of the first two reaction vessels is divided into two vessels yielding two pairs of vessels. For each pair, a parent codon is synthesized in one of the vessels and a random codon is synthesized in the second vessel. When arranged linearly, the reaction products in the second and third vessels are mixed to bring together those products having random codon sequences at single codon positions. This mixing also reduces the product populations to three, which are the starting populations for the next round of synthesis. Similarly, for the third, fourth and each remaining position, each reaction product population for the preceding position are divided and a parent and random codon synthesized.

Following the above modification of codon-based synthesis, populations containing random codon changes at one, two, three, and four positions as well as others can be conveniently separated out and used based on the needs of the individual. Moreover, this synthesis scheme also allows enrichment of the populations for the randomized sequences over the parent sequence since the vessel containing only the parent sequence synthesis is similarly separated out from the random codon synthesis.

Other well known art methods for producing a large number of alterations in a known amino acid sequence or for generating a diverse population of variable or random sequences include, for example, degenerate or partially degenerate oligonucleotide synthesis. Codons specifying equal mixtures of all four nucleotide monomers, represented as NNN, result in degenerate synthesis, whereas partially degenerate synthesis can be accomplished using, for example, the NNG/T codon previously described. Other well known art methods can alternatively be used such as, for example, the use of statistically predetermined that varigated, codon synthesis is the subject matter of U.S. Pat. Nos. 5,223,409 and 5,403,484.

Once the populations of altered variable region encoding nucleic acids have been constructed as described , they can be expressed to generate a population of altered variable region polypeptides that can be screened for binding affinity. For example, the altered variable region encoding nucleic acids can be cloned into an appropriate vector for propagation, manipulation, and expression. Such vectors are known or can be constructed by those skilled in the art and should contain all expression elements sufficient for the transcription, translation, regulation, and if desired, sorting, and secretion of the altered variable region polypeptides. The vectors also can be employed for use in either procaryotic or eukaryotic host systems so long as the expression and regulatory elements are of compatible origin. Additionally, the expression vectors can include regulatory elements for inducible or cell type-specific expression. One skilled in the art will know which host systems are compatible with a particular vector and which regulatory or functional elements are sufficient to achieve expression of the polypeptides in soluble, secreted, or cell surface forms.

Appropriate host cells, include, for example, bacteria and corresponding bacteriophage expression systems, yeast, avian, insect, and mammalian cells. Methods for recombinant expression, screening, and purification of populations of altered variable regions or altered variable region polypeptides within such populations in various host systems are well known in the art and are described, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998). The choice of a particular vector and host system for expression and screening of altered variable regions will be known by those skilled in the art and will depend on the preference of the user. A specific example of the expression of recombinant altered variable region polypeptides is additionally described below in the example section.

Moreover, expression of diverse populations of hetereomeric receptors in either soluble or cell surface form using filamentous bacteriophage vector/host systems is well known in the art and is the subject matter of U.S. Pat. No. 5,871,974.

The expressed population of altered variable region polypeptides can be screened for the identification of one or more altered variable region species exhibiting binding affinity that is substantially the same or greater than the donor CDR variable region. Screening can be accomplished using various methods well known in the art for determining the binding affinity of a polypeptide or compound. Additionaly, methods based on determining the relative affinity of binding molecules to their partner by comparing the amount of binding between the altered variable region polypeptides and the donor CDR variable region can be used similarly for the identification of species exhibiting binding affinity that is substantially the same or greater than the donor CDR variable region. All such methods can be performed, for example, in solution or in solid phase. Moreover, various binding assay formats are well known in the art and include, for example, immobilization to filters such as nylon or nitrocellulose; two-dimensional arrays; enzyme linked immunosorbant assay (ELISA); radioimmune assay (RIA); panning; and plasmon resonance. Such methods are described, for example, in Sambrook, et al., supra, and Ansubel, et al.

For the screening of populations of polypeptides such as the altered variable region populations produced by the methods of the invention, immobilization of the populations of altered variable regions to filters or other solid substrate is particularly advantageous because large numbers of different species can be efficiently screened for antigen binding. Such filter lifts will allow for the identification of altered variable regions that exhibit substantially the same or greater binding affinity compared to the donor CDR variable region. Alternatively, if the populations of altered variable regions are expressed on the surface of a cell or bacteriophage, for example, panning on immobilized antigen can be used to efficiently screen for the relative binding affinity of species within the population and for those which exhibit substantially the same or greater binding affinity than the donor CDR variable region.

Another affinity method for screening populations of altered variable regions polypeptides is a capture lift assay that is useful for identifying a binding molecule having selective affinity for a ligand (Watkins et. al., (1997)). This method employs the selective immobilization of altered variable regions to a solid support and then screening of the selectively immobilized altered variable regions for selective binding interactions against the cognate antigen or binding partner. Selective immobilization functions to increase the sensitivity of the binding interaction being measured since initial immobilization of a population of altered variable regions onto a solid support reduces non-specific binding interactions with irrelevant molecules or contaminants that can be present in the reaction.

Another method for screening populations or for measuring the affinity of individual altered variable region polypeptides uses surface plasmon resonance (SPR), a method based on the phenomenon that occurs when surface plasmon waves are excited at a metal/liquid interface, for example, light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously to allow for determination of the individual components of an affinity constant. More specifically, the method enables accurate measurements of association rates (kon) and disassociation rates (koff).

Measurements of kon and koff values can be advantageous because they can identify altered variable regions or optimized variable regions that are therapeutically more efficacious. For example, an altered variable region, or heteromeric binding fragment thereof, can be more efficacious because it has, for example, a higher kon valued compared to variable regions and heteromeric binding fragments that exhibit similar binding affinity. Increased efficacy is conferred because molecules with higher kon values can specifically bind and inhibit their target at a faster rate. Similarly, a molecule of the invention can be more efficacious because it exhibits a lower koff value compared to molecules having similar binding affinity. Increased efficacy observed with molecules having lower koff rates can be observed because, once bound, the molecules are slower to dissociate from their target. Although described with reference to the altered variable regions and optimized variable regions of the invention including, heteromeric variable region binding fragments thereof, the methods described above for measuring association and disassociation rates are applicable to essentially any antibody or fragment thereof for identifying desirable characteristics (e.g., more effective binders) for therapeutic or diagnostic purposes.

Methods for measuring the affinity, including association and disassociation rates using surface plasmon resonance are well known in the art and can be found described in, for example, Jönsson and Malmquist, Advances in Biosnsors, 2:291-336 (1992) and Wu et al. Proc. Natl. Acad. Sci. USA, 95:6037-6042 (1998). Moreover, one apparatus well known in the art for measuring binding interactions is a BIAcore 2000 instrument, which is commercially available through Pharmacia Biosensor, (Uppsala, Sweden).

Using any of the above described screening methods, as well as others well known in the art, an altered variable region having binding affinity substantially the same or greater than the donor CDR variable region is identified by detecting the binding of at least one altered variable region within the population to its antigen or cognate ligand. Additionally, the above methods can alternatively be modified by, for example, the addition of substrate and reactants, to identify using the methods of the invention, altered variable regions having catalytic activity substantially the same or greater that the donor CDR variable region within the populations. Comparision, either independently or simultaneously in the same screen, with the donor variable region will identify those binders that have substantially the same or greater binding affinity as the donor. Those skilled in the art will know, or can determine using the donor variable region, binding conditions that are sufficient to identify selective interactions over non-specific binding.

Detection methods for identification of binding species within the population of altered variable regions can be direct or indirect and can include, for example, the measurement of light emission, radioisotopes, calorimetric dyes, and fluorochromes. Direct detection includes methods that operate without intermediates or secondary measuring procedures to assess the amount of bound antigen or ligand. Such methods generally employ ligands that are themselves labeled by, for example, radioactive, light emitting, or fluorescent moieties. In contrast, indirect detection includes methods that operate through an intermediate or secondary measuring procedure. These methods generally employ molecules that specifically react with the antigen or ligand and can themselves be directly labeled or detected by a secondary reagent. For example, an antibody specific for a ligand can be detected using a secondary antibody capable of interacting with the first antibody specific for the ligand (again using the detection methods described above for direct detection). Indirect methods can additionally employ detection by enzymatic labels. Moreover, for the specific example of screening for catalytic antibodies, the disappearance of a substrate or the appearance of a product can be used as an indirect measure of binding affinity or catalytic activity.

Isolated variable regions exhibit binding affinity as single chains, in the absence of assembly into a heteromeric structure with their respective VH or VL subunits. As such, populations of VH and VL altered variable regions polypeptides can be expressed alone and screened for binding affinity having substantially the same or greater binding affinity compared to the CDR donor VH or VL variable region. Alternatively, polypeptide populations of VH and VL altered variable regions polypeptides can be coexpressed so that they self-assemble into heteromeric altered variable region binding fragments. The heteromeric binding fragment population can then be screened for species exhibiting binding affinity substantially the same or greater than the CDR donor variable region binding fragment. A specific example of the coexpression and self-assembly of populations VH and VL altered variable regions into heteromeric populations is described further below in the Example Section.

Therefore, the invention provides a method of simultaneously grafting and optimizing the binding affinity of a variable region binding fragment. The method consists of: (a) constructing a population of altered heavy chain variable region encoding nucleic acids consisting of an acceptor variable region framework, containing donor CDRs and a plurality of different amino acids at one or more framework regions and CDR amino acid positions; (b) coexpressing the populations of heavy and light chain variable region encoding nucleic acids to produce diverse combinations of heteromeric variable region binding fragments, and (c) identifying one or more heteromeric variable region binding fragments having affinity substantially the same (or greater than) the donor CDR heteromeric variable region binding fragment.

The invention additionally provides a method of optimizing the binding affinity of an antibody variable region. The method consists of: (a) constructing a population of antibody variable region encoding nucleic acids, said population comprising two or more CDRs containing a plurality of different amino acids at one or more CDR amino acid positions; (b) expressing said population of variable region encoding nucleic acids, and (c) identifying one or more variable regions having binding affinity substantially the same or greater than the donor CDR variable region.

The methods described above, for conferring donor CDR binding affinity onto an antibody acceptor variable region framework and for simultaneously grafting and optimizing the binding affinity of a heteromeric variable region binding fragment, can additionally be employed to modify or optimize the binding affinity of a variable region or a heteromeric variable region binding fragment. Similar to the previously described methods, the method for modifying or optimizing binding affinity involves the selection of relevant amino acid positions and the construction, expression, and screening of variable region populations containing variable amino acid residues at all or a fraction of the selected positions. However, for optimization of binding affinity it is not necessary to vary amino acid positions in the framework regions. Instead, all that is required is to alter one or more amino acid positions in two or more CDR regions. Changing the CDR amino acid residues directly effects the binding affinity. Once a population containing variable amino acid residues incorporated in two or more CDRs is produced, all that is necessary is to screen the population for species that contain the desired binding affinity modification. All of the criteria for selecting relevant amino acid positions described previously are applicable for use in this mode of the method. Therefore, the methods for modifying or optimizing the binding affinity of a variable region or a heteromeric variable region binding fragment by altering one or more amino acid positions in two or more CDR regions are applicable to essentially any variable region, grafted variable region as well as applicable to the altered and/or optimized variable regions of the invention.

Moreover, by incorporating variable amino acid residues in two or more CDRs when employing the methods conferring donor CDR binding affinity onto an acceptor framework, this method of modifying binding affinity is therefore useful for simultaneously optimizing the binding affinity of a grafted antibody. Employing the methods for simultaneously grafting and optimizing, or for optimizing, it is possible to generate heteromeric variable region binding fragments having increases in affinities of greater than 5-, 8-, and 10-fold. In particular, heteromeric variable region binding fragments can be generated having increases in affinities of greater than 12-, 15-, 20-, and 25-fold as well as affinities greater than 50-, 100-, and 1000-fold compared to the donor or parent molecule.

As mentioned above, for optimization of binding affinity, it is not necessary to vary amino acid positions in the framework. Prior to the present work, it was believed that at least some of acceptor framework amino acids had to be changed to donor (e.g. murine) amino acids to maintain the binding affinity of the donor CDRs. As disclosed herein, the present invention teaches that the acceptor framework (e.g. human framework) does not need to be modified to maintain donor CDR binding affinity. Instead, the framework may remain unvaried compared to the parental frameworks, while the CDRs are modified to retain, and preferably optimize (e.g. increase) the donor CDR activity (e.g. antigen binding affinity, on-rate, off-rate, etc.). In this regard, optimized heteromeric variable region binding fragments exhibiting optimized activity compared to a donor (e.g. non-human) heteromeric variable region binding fragment may be generated, where the optimized heteromeric variable region binding fragment has altered light and heavy chain variable regions. Preferably, the altered light chain variable region comprises four unvaried light chain framework regions (e.g. human germline), and three light chain altered variable region CDRs, with a least one of the light chain altered variable region CDRs being a light chain donor CDR variant (i.e., the light chain donor CDR variant comprises a different amino acid at one or more positions when compared to the corresponding light chain donor CDR). It is also preferred that the altered heavy chain variable region comprises four unvaried heavy chain framework regions (e.g. human germline), and three heavy chain altered variable region CDRs, with a least one of the heavy chain altered variable region CDRs being a heavy chain donor CDR variant (i.e., the heavy chain donor CDR variant comprises a different amino acid at one or more positions when compared to the corresponding heavy chain donor CDR). In certain preferred embodiments, the donor heteromeric variable region is non-human (e.g., murine) and the unvaried light and heavy chain framework regions are human (e.g., human germline framework regions).

In selecting an unvaried framework (e.g., human germline), in some embodiments, a number of criteria may be employed (although employing selection criteria is not necessary to understand or practice the present invention). For example, one may select frameworks that are known to be less immunogenic for a particular host (e.g. human). Also, for example, one may compare key amino acids in the donor framework with the proposed unvaried frameworks, and select the unvaried framework that is most similar to the donor framework at the key amino acid positions (e.g., amino acid positions that are known to be associated with characteristic loop conformations or based on structural modeling). Another criteria that can be employed is to use an unvaried framework that has CDRs that are approximately the same size (length) as the donor framework CDRs. An additional criteria that may be used (e.g., for humans) to minimize the risk of immunogenicity, is to eliminate human genes that are non-functional or that are infrequently used in the human population (i.e., select human frameworks that are frequently used in the human population).

One advantage of the present invention is the fact that unvaried framework regions may be employed. Preferably, the unvaried frameworks are human frameworks. In particularly preferred embodiments, the frameworks are human germline sequences. For example, the NCBI web site contains the sequences for the currently known human framework regions. Examples of human VH sequences include, but are not limited to, VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81, which are provided in Matsuda et al., (1998) J. Exp. Med. 188:1973-1975, that includes the complete nucleotide sequence of the human immunoglobulin chain variable region locus, herein incorporated by reference. Examples of human VK sequences include, but are not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8, which are provided in Kawasaki et al., (2001) Eur. J. Immunol. 31:1017-1028; Schable and Zachau, (1993) Biol. Chem. Hoppe Seyler 374:1001-1022; and Brensing-Kuppers et al., (1997) Gene 191:173-181, all of which are herein incorporated by reference. Examples of human VL sequences include, but are not limited to, V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6, which are provided in Kawasaki et al., (1997) Genome Res. 7:250-261, herein incorporated by reference. Unvaried human frameworks can be selected from any of these functional germline genes.

While not necessary to practice or understand the invention, it is believed that the use of germline frameworks is expected to help eliminate or reduce adverse immune responses (e.g., when administered therapeutically) in most individuals. Somatic mutations frequently occur in the variable region of immunoglobulins as a result of the affinity maturation step that takes place during a normal immune response. Although these mutations are predominantly clustered around the hypervariable CDRs, they also impact residues in the framework regions. These framework mutations are not present in the germline genes and therefore such framework mutations may be immunogenic when administered to patients. In contrast, the general population has been exposed to the vast majority of framework sequences expressed from germline genes and, as a result of immunologic tolerance, these germline frameworks should be less, or non-immunogenic in patients. To maximize the likelihood of tolerance, genes encoding the variable regions can be selected from a collection of commonly occurring, functional germline genes, and genes encoding VH and VL regions can further be selected to match known associations between specific heavy and light chains of immunoglobulin molecules. Also, germline variable regions genes that are frequently utilized in the human population are preferred (again, to limit the likelihood of adverse immunogenic reactions). For example, the human kappa light chain germline gene that is the source of the light chain framework may be selected from the following; A11, A17, A18, A19, A20, A27, A30, L1, L11, L12, L2, L5, L6, L8, O12, O2, and O8. Likewise, the human heavy chain germline gene that is the source of the heavy chain framework may be selected from the following VH2-5, VH2-26, VH2-70, VH3-20, VH3-72, VH1-46, VH3-9, VH3-66, VH3-74, VH4-31, VH1-18, VH1-69, VH3-7, VH3-11, VH3-15, VH3-21, VH3-23, VH3-30, VH3-48, VH4-39, VH4-59, and VH5-51.

Additionally, the methods described herein for optimizing are also applicable for producing catalytic heteromeric variable region fragments or for optimizing their catalytic activity. Catalytic activity can be optimized by changing, for example, the on or off rate, the substrate binding affinity, the transition state binding affinity, the turnover rate (kcat) or the Km. Methods for measuring these characteristics are well known in the art. Such methods can be employed in the screening steps of the methods described above when used for optimizing the catalytic activity of a heteromeric variable region binding fragment.

The methods for conferring donor CDR binding affinity onto an antibody acceptor variable region framework described previously are applicable for use with essentially any distinguishable donor and acceptor pair. Many applications of the methods will be for the production and optimization of variable region binding fragments having human acceptor frameworks due to the therapeutic importance of such molecules in the treatment of human diseases. However, the methods are applicable for conferring donor CDR binding affinity onto an acceptor originating from the same or a divergent species as the CDR donor variable region so long as the framework regions between the donor and acceptor variable regions are distinct. Therefore, the invention encompasses altered variable regions having acceptor frameworks derived, for example, from human, mouse, rat, rabbit, goat, and chicken, for example.

Additionally, the methods for conferring donor CDR binding affinity onto an antibody acceptor variable region framework are applicable for grafting CDRs as described by Kabat, et al., supra, Chothia, et al., supra, or MacCallum, et al., supra. The methods similarly can be used for grafting into an acceptor framework overlapping regions or combinations of CDR as described by these authors. Generally, the methods will graft variable region CDRs by identifying the boundries described by one of the CDR definitions known in the art and set forth herein. However, because the methods are directed to constructing and screening populations of CDR grafted altered variable regions which incorporate relevant amino acid position changes in both the framework and CDR regions, and such variations can, for example, compensate or augment amino acid changes elsewhere in the variable region, the exact boundry of a particular CDR or set of variable region CDRs can be varied. Therefore, the exact CDR region to graft, whether it is the region described by Kabat, et al., Chothia, et al., or MacCallum, et al., or any combination thereof, will essentially depend on the preference of the user.

Similarly, the methods described previously for optimizing the binding affinity of an antibody also are applicable for use with essentially any variable region for which an encoding nucleic acid is or can be made available. As with the methods for conferring donor CDR binding affinity, many applications of the methods for optimizing binding affinity will be for modifying the binding affinity of CDR grafted variable regions having human frameworks. Again, such molecules are significantly less antigenic in human patients and therefore, therapeutically valuable in the treatment of human diseases. However, the methods of the invention for optimizing the binding affinity of a variable region are applicable to all species of variable regions. Therefore, the invention includes binding affinity optimization of variable regions derived from human, mouse, rat, rabbit, goat, and chicken, for example.

The methods of the invention have been described with reference to variable regions and heteromic variable region binding fragments. Given these descriptions and teachings herein, those skilled in the art will understand that all of such methods are applicable to whole antibodies and functional fragments thereof as well as to regions and functional domains other than the antigen binding variable region of antibodies. Moreover, the methods described herein are further applicable to molecules other than antibodies, variable regions, and other antibody functional domains. Given the teachings of the invention, those skilled in the art will know how to apply the methods of simultaneously constructing hybrid molecules and maintaining or optimizing the binding affinity or catalytic activity of a target molecule, as well as how to apply the methods of optimizing the binding affinity or catalytic activity to a variety of different types and classes of polypeptides and proteins.

The methods for optimizing the binding affinity of an antibody variable region can include the selection of relevant acceptor framework and donor CDR amino acid positions to be altered. Amino acid residues selected for alteration during binding affinity optimization are typically amino positions predicted to be relatively important for structure and/or function. Criteria that can be used for identifying amino positions to be altered include, for example, conservation of amino acids among polypeptide subfamily members and/or knowledge that particular amino acids are predicted to be important in polypeptide conformation and/or structure, as described herein. Alternatively, potentially important framework residues that differ between acceptor framework and donor CDR can be characterized without structural information by synthesizing and expressing a combinatorial antibody library that contains all possible combinations of amino acids in framework positions to be optimized.

The invention provides a method for identifying one or more functional amino acid positions of a polypeptide. The method consists of (a) constructing a population of nucleic acids encoding a population of altered polypeptides containing substitutions of one or more amino acid positions within a polypeptide; (b) expressing the population of nucleic acids; (c) identifying nucleic acids encoding altered polypeptides having a functional activity of the polypeptide; (d) sequencing a subset of nucleic acids encoding altered polypeptides having a functional activity, and (e) comparing an amino acid position in a polypeptide corresponding to an amino acid position in the subset of altered polypeptides wherein an amino acid position exhibiting a biased representation of amino acid residues indicates a functional amino acid position in the polypeptide.

The method of the invention directed to identifying a functional amino acid position in a polypeptide involves substituting one or more amino acid positions in a polypeptide with a plurality of amino acid residues, as described previously for optimizing the binding affinity of an antibody, and identifying altered polypeptides having an activity that is substantially the same or greater than the parent polypeptide. Functional amino acid positions identified using the methods of the invention are amino acid positions important for a conformation, functional activity, or structure of a polypeptide. Functional activities of a polypeptide can include, for example, binding affinity to a substrate, ligand, or other interacting molecule, and catalytic activity.

The identification of functional amino acid positions in a polypeptide involves constructing a population of nucleic acids encoding a population of altered polypeptides containing amino acid substitutions at specific amino acid positions. Substituted amino acids include all twenty naturally occurring amino acid residues or a subset of amino acid residues, as described previously in detail. Nucleic acid populations can be constructed by any method known in the art and as described previously. A population of nucleic acids encoding altered polypeptides is expressed in an appropriate host cell, and a functional activity of altered polypeptides is detected and compared with that of the polypeptide. Any method known in the art that is appropriate for determining a polypeptide functional activity can be used to compare polypeptide and altered polypeptide functional activities.

A subset of nucleic acids encoding altered polypeptides having a functional activity that is substantially the same or greater than that of the polypeptide is sequenced.

A subset can include a few molecules to many members constituting the population of nucleic acids encoding altered polypeptides. For example, a subset can consist of about 2-5, 6-10, 10-20, and 21 or greater members of the population. The actual number sequenced will vary with the total size of the nucleic acid population. Generally, however, a subset of about 15-25 and typically about 20 members is sufficient to identify functional amino acids.

Amino acid residues at substituted positions in the polypeptide are compared to the corresponding position in altered polypeptides. An amino acid position that contains the same amino acid or a conservative substitution among the population of altered polypeptides exhibits biased representation of that amino acid residue. Biased representation indicates that a particular amino acid is required for polypeptide function. Amino acid positions that are biased are therefore considered important for functional activity of a polypeptide. Amino acid positions that contain a variety of substituted amino acids are unbiased and considered not important or less important for a polypeptide function.

The method of identifying an amino acid position important for polypeptide function is useful for a variety of applications, such as, for example, the determination of a consensus sequence of amino acids important for a polypeptide functional activity. A consensus sequence is useful for the optimization of a polypeptide function because amino acid positions determined to be important for functional activity can be unaltered while amino acid positions not important for activity can be varied. Polypeptide functions that can be optimized using the method of the invention include, for example, catalytic activity, polypeptide conformation, and binding affinity.

The identification of a functional amino acid position in a polypeptide can be applied to determining a consensus sequence of amino acids that impart a particular activity to a polypeptide. For example, a consensus sequence that provides a catalytic activity to an enzyme can be determined using the methods of the invention. To identify amino acid positions that are important or critical to catalytic activity of an enzyme, one or more of amino acid positions are substituted with a plurality of amino acid substitutions, as described previously. A nucleic acid population encoding altered enzyme polypeptides is constructed and expressed in host cells. The catalytic activity of altered enzymes is measured and compared with a parent enzyme or other catalytically active form of the enzyme.

Nucleic acids encoding a subset of altered enzyme polypeptides identified by functional activity are sequenced, and the amino acid sequences of altered polypeptides are compared. Amino acid positions that contain a particular amino acid or a conservative substitution are determined to be important for a catalytic activity of the enzyme. A sequence of amino acids determined to be biased in a polypeptide can thus provide a consensus sequence that defines amino acid positions required for catalytic activity. A consensus sequence of residues important for various aspects of catalytic activity such as, for example, substrate binding, proper active site conformation, and co-factor binding can be identified using the methods of the invention by measuring enzyme catalytic activity, as described above.

Similarly, a consensus sequence associated with a particular conformation of a polypeptide can be determined using the method of the invention in essentially the same manner as described above for polypeptide catalytic activity. The amino acid positions that have functional roles in a polypeptide conformation can be determined so long as a particular conformation state can be detected and compared between a polypeptide and an altered polypeptide. For example, a consensus sequence of a polypeptide conformation that confers a particular functional activity to a polypeptide or a particular structural feature to a polypeptide can be determined using the methods of the invention. A structural feature can include, for example, the exposure of a certain amino acid on the surface of a polypeptide.

A consensus sequence of amino acid positions in a polypeptide important for binding affinity can also be determined using the methods of the invention. The binding affinities of polypeptides include, for example, the binding affinity between two or more polypeptides in a protein-protein interaction and the binding affinity between a polypeptide and a substrate. For example, a consensus sequence for the binding affinity of an antibody for an antigen can be determined, and can be applied to the process of antibody humanization.

The identification of a functional amino acid position in a polypeptide can be applied to determining the consensus sequence for a humanized version of an antibody that preserves the binding activity of the parent antibody. For example, a library containing all possible combinations of human template and murine parent antibody residues in a selected number of amino acid residue positions can be synthesized by any method known in the art, for example, using codon-based mutagenesis as described. Framework polypeptides containing amino acid substitutions can then be screened by functional binding to identify altered framework polypeptides that have a binding affinity substantially the same as the parent antibody. Of the amino acid positions altered, only a small percentage of framework positions are typically critical for antibody binding activity. Therefore, a low throughput screening method of identifying active humanized framework variants can be used. Sequencing of nucleic acids encoding humanized frameworks displaying a functional activity of the parent antibody is then used to identify altered polypeptides having significant bias toward murine human residues. Thus, a consensus humanization sequence for maintaining full binding activity of an antibody can be prepared by using murine CDRs grafted onto a human template on which amino acid positions are changed to the corresponding residue determined to be important for binding activity.

In certain embodiments, the present invention provides binding molecules (e.g. heteromeric variable region binding fragments and antibodies) that are able to bind von Willebrand factor (vWF) (preferably human vWF). In some embodiments, these vWF binding molecules comprises an unvaried human framework. In particular embodiments, the unvaried human framework is a human germline framework. Exemplary CDRs useful for generating such vWF binding molecules are shown in tables 4 and 5.

As described below in tables 4 and 5, the present invention provides numerous CDRs useful for generating vWF binding molecules. For example, one or more of the CDRs shown in tables 4 and 5 (and Example 2) can be combined with a framework sub-region (e.g., a fully human FR1, FR2, FR3, or FR4) to generate a vWF binding molecule, or a nucleic acid sequence encoding a vWF binding molecule. Also, the CDRs shown in the tables below may be combined, for example, such that three CDRs are present in a light chain variable region, and/or three CDRs are present in a heavy chain variable region. The CDRs shown below may be inserted into a human framework (e.g., by recombinant techniques) to generate vWF binding molecules or nucleic acid sequences encoding vWF binding molecules.

TABLE 4
Light Chain CDRs

| | CDR | |
|---|---|---|
| SEQ ID NO | Name* | Sequence |
| SEQ ID NO:9 | CDRL1 | SASQDINDYLN |
| SEQ ID NO:10 | CDRL1 | AGTGCAAGTCAGGACATTAACGACTATTTAA |
| | | AC |
| SEQ ID NO:11 | CDRL2 | GTSSLHS |
| SEQ ID NO:12 | CDRL2 | GGCACATCAAGTTTACACTCA |
| SEQ ID NO:13 | CDRL2 | NTSSLHS |
| SEQ ID NO:14 | CDRL2 | AACACATCAAGTTTACACTCA |
| SEQ ID NO:15 | CDRL2 | YTSVLHS |
| SEQ ID NO:16 | CDRL2 | TACACATCAGTTTTACACTCA |
| SEQ ID NO:17 | CDRL2 | NTSVLHS |
| SEQ ID NO:18 | CDRL2 | AACACATCAGTTTTACACTCA |
| SEQ ID NO:19 | CDRL2 | YTSSLHV |
| SEQ ID NO:20 | CDRL2 | TACACATCAAGTTTACACGTG |
| SEQ ID NO:21 | CDRL2 | NTSSLHV |
| SEQ ID NO:22 | CDRL2 | AACACATCAAGTTTACACGTA |
| SEQ ID NO:23 | CDRL3 | QQYEDLPWT |
| SEQ ID NO:24 | CDRL3 | CAGCAGTATGAAGATCTTCCGTGGACG |

*The work of Kabat was used to number residues. CDRs include Kabat and Chothia residues.

TABLE 5
Heavy Chain CDRs

| | CDR | |
|---|---|---|
| SEQ ID NO | Name* | Sequence |
| SEQ ID NO:25 | CDRH1 | GFSLGDYGVD |
| SEQ ID NO:26 | CDRH1 | GGATTCTCATTAGGCGACTATGGTGTAGAC |
| SEQ ID NO:27 | CDRH2a | MIWPDGST |
| SEQ ID NO:28 | CDRH2a | ATGATATGGCCGGATGGAAGCACA |
| SEQ ID NO:29 | CDRH2a | MIWQDGST |
| SEQ ID NO:30 | CDRH2a | ATGATATGGCAGGATGGAAGCACA |
| SEQ ID NO:31 | CDRH2a | MIWGDGSV |
| SEQ ID NO:32 | CDRH2a | ATGATATGGGGTGATGGAAGCGTA |
| SEQ ID NO:33 | CDRH2b | DINSALKS |
| SEQ ID NO:34 | CDRH2b | GACATTAATTCAGCTCTCAAGTCC |
| SEQ ID NO:35 | CDRH2b | DYNSALAS |
| SEQ ID NO:36 | CDRH2b | GACTATAATTCAGCTCTCGCATCC |
| SEQ ID NO:37 | CDRH2b | DYNSALQS |
| SEQ ID NO:38 | CDRH2b | GACTATAATTCAGCTCTCCAATCC |
| SEQ ID NO:39 | CDRH2b | DVNSALQS |
| SEQ ID NO:40 | CDRH2b | GACGTTAATTCAGCTCTCCAGTCC |
| SEQ ID NO:41 | CDRH2b | DVNSALKS |
| SEQ ID NO:42 | CDRH2b | GACGTTAATTCAGCTCTCAAGTCC |
| SEQ ID NO:43 | CDRH3 | DPADYGNYNYALDY |
| SEQ ID NO:44 | CDRH3 | GACCCAGCCGACTATGGTAACTACAATTATG |
| | | CTTTGGACTAC |
| SEQ ID NO:45 | GDRH3 | DWADYGNYNYALDY |
| SEQ ID NO:46 | CDRH3 | GACTGGGCCGACTATGGTAACTACAATTATG |
| | | CTTTGGACTAC |
| SEQ ID NO:47 | CDRH3 | DPADYGNYDYKLDY |
| SEQ ID NO:48 | CDRH3 | GACCCAGCCGACTATGGTAACTACGATTATA |
| | | AATTGGACTAC |
| SEQ ID NO:49 | CDRH3 | DWADYGNYDYALDY |
| SEQ ID NO:50 | CDRH3 | GACTGGGCCGACTATGGTAACTACGACTATG |
| | | CTTTGGACTAC |

The present invention also provides sequences that are substantially the same as the CDR sequences (both amino acid and nucleic acid) shown in the above Tables. For example, one or two amino acid may be changed in the sequences shown in the Tables. Also for example, a number of nucleotide bases may be changed in the sequences shown in the Tables (e.g. based on the degeneracy of the genetic code, such that the same peptide is still encoded by the nucleic acid sequence). Changes to the amino acid sequence may be generated by changing the nucleic acid sequence encoding the amino acid sequence. A nucleic acid sequence encoding a variant of a given CDR may be prepared by methods known in the art. These methods include, but are not limited to, preparation by site-directed (or oligonucleotide-mediated) mutagenesis, PCR mutagenesis, and cassette mutagenesis of an earlier prepared nucleic acid encoding the CDR. Site-directed mutagenesis is a preferred method for preparing substitution variants. This technique is well known in the art (see, e.g., Carter et al., (1985) Nucleic Acids Res. 13: 4431-4443 and Kunkel et. al., (1987) Proc. Natl. Acad. Sci. USA 82: 488-492, both of which are hereby incorporated by reference).

It is understood that modifications which do not substantially affect the activity of the various embodiments of this invention are also included within the definition of the invention provided herein. Accordingly, the following examples are intended to illustrate but not limit the present invention.

### EXAMPLE I

### Simultaneous Humanization and Affinity Maturation of an Anti-CD40 Antibody

This example shows the simultaneous humanization and affinity maturation of the murine mAb 40.2.220, directed against the CD40 receptor.

The CD40 receptor is a potential therapeutic target for several diseases. For example, the interaction of the CD40 receptor and its ligand, gp39, serves a critical role in both humoral and cell-mediated immune responses (Foy et al., Annu. Rev. Immunol., 14:591-616, 1996). Immunological rejection of organs from genetically non-identical individuals, termed graft-versus-host-disease (GVHD), is mediated through T cell-dependent mechanisms. In vivo administration of an anti-gp39 mAb blocks GVHD in mice and inhibits many of the GVHD-associated phenomena (Durie et al., J. Clin. Invest., 94:1333-38, 1994), providing evidence that the CD40/gp39 interaction plays a critical role in the development of GVHD. More recently, inhibition of the CD40/gp39 interaction in vivo in hyperlipidemic mice fed a high cholesterol diet limited atherosclerosis, suggesting that CD40 signalling may also play a role in atherogenesis (Mach et. al., Nature 394:200-203, 1998). In addition, the CD40 receptor is overexpressed on hematologic malignancies (Uckun et al., Blood, 76:2449-56, 1990) and certain carcinomas (Stamenkovic et al., EMBO J., 8:1403-10, 1989) and thus, may serve as a target for cytotoxic agents. An anti-CD40 single chain antibody-toxin fusion was cytotoxic against CD40-expressing malignant cells in vitro (Francisco et al., Cancer Res., 55:3099-3104, 1995) and was efficacious in treating human non-Hodgkin's lymphoma xenografted SCID mice (Francisco et. al., Blood, 89:4493-4500, 1997).

Codon-based mutagenesis (Glaser et. al., J. Immunol., 149:3903-3913, 1992) was used to create libraries of LCDR3, HCDR3 and framework region variants of mAb 40.2.220 sequences. Libraries composed of framework region variants alone and in combination with HCDR3 variants and with HCDR3 and LCDR3 variants together were screened for high affinity variants. It was demonstrated that in combination higher affinity variants were obtained than those obtained when codon-based mutagenesis was applied independently thus showing (1) higher affinity variants that could only be obtained by the use of codon-based mutagenesis simultaneously on disparate regions of the mAb and (2) the use of codon-based mutagenesis to uncover potential direct interactions between disparate regions of a mAb.

A vector for the production of a chimeric anti- CD40 murine mAb 40.2.220 was constructed. Based on the sequence of anti-CD40 murine mAb 40.2.220 (provided by Dr. D. Hollenbaugh, Bristol-Myers Squibb, Princeton, N.J.) overlapping oligonucleotides encoding VH and VL (69-75 bases in length) were synthesized and purified. The variable H and L domains were synthesized separately by combining 25 pmol of each of the overlapping oligonucleotides with Pfu DNA polymerase (Stratagene) in a 50 μl PCR reaction consisting of 5 cycles of: denaturing at 94° C. for 20 sec, annealing at 50° C. for 30 sec, ramping to 72° C. over 1 min, and maintaining at 72° C. for 30 sec. Subsequently, the annealing temperature was increased to 55° C. for 25 cycles. A reverse primer and a biotinylated forward primer were used to further amplify 1 μl of the fusion product in a 100 μl PCR reaction using the same program. The products were purified by agarose gel electrophoresis, electroeluted, and phosphorylated by T4 polynucleotide kinase (Boehringer Mannheim) and were then incubated with streptavidin magnetic beads (Boehringer Mannheim) in 5 mM Tris-Cl, pH 7.5, 0.5 mM EDTA, 1 M NaCl, and 0.05% Tween 20 for 15 min at 25° C. The beads were washed and the non-biotinylated, minus strand DNA was eluted by incubating with 0.15 M NaOH at 25° C. for 10 min. Chimeric anti-CD40 Fab was synthesized in a modified M131X104 phage vector (Kristensson et. al., 1995, Vaccines 95, pages 39-43, Cold Spring Harbor Labs, Cold Spring Harbor, N.Y.), termed M131X104CS, by hybridization mutagenesis (Rosok et. al., JBC, 271:22611-18, 1996); Kunkel, PNAS, 82:488-92,1985) using the VH and VL oligonucleotides in 3-fold molar excess of the uridinylated vector template. The M131X104 vector was modified by replacing cysteine residues at the end of the kappa and 1 constant regions with serine. The reaction was electroporated into DH10B cells and titered onto a lawn of XL-1 Blue.

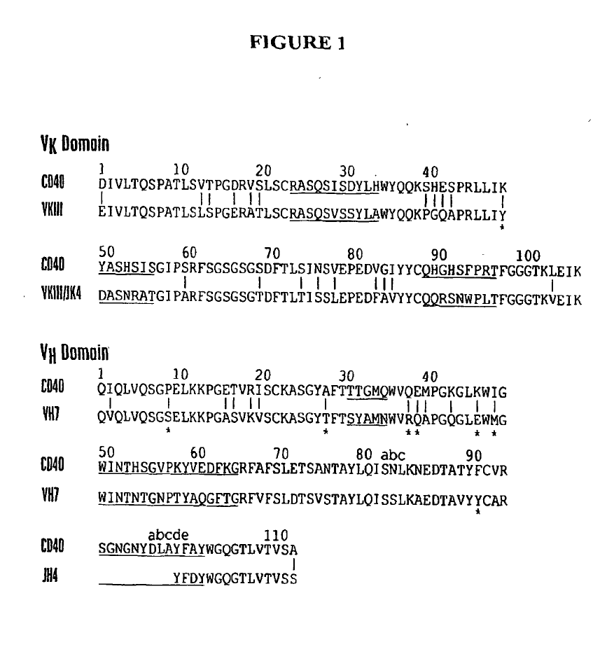

The murine anti-CD40 mAb variable region framework sequences were used to identify the most homologous human germline sequences. The H chain framework residues were 74% identical to human germline VH7 (7-4.1) and JH4 sequences while the L chain was 75% identical to the corresponding human germline VKIII (L6) and JK4 sequences. Alignment of the H and L chain variable sequences is shown in FIG. 1. CDR residues, as defined by Kabat et. al. (1977, 1991), are underlined and were excluded from the homology analysis. Differences in sequence are indicated by vertical lines and framework positions characterized in the combinatorial expression library are marked with an asterisk. Framework residues that differed between the murine mAb and the human templates were assessed individually.

Based on structural and sequence analysis, antibody CDRs with the exception of HCDR3 display a limited number of main chain conformations termed canonical structures (Chothia & Lesk, (1987); Chothia et. al., (1989)). Moreover, certain residues critical for determining the main chain conformation of the CDR loops have been identified (Chothia & Lesk, (1987); Chothia et. al., (1989)). Canonical framework residues of murine anti-CD40 were identified therefore, and it was determined that amino acids at all critical canonical positions within the H and L chain frameworks of the human templates were identical to the corresponding murine residues.

Surface-exposed murine amino acids not normally found in human antibodies are likely to contribute to the immunogenicity of the humanized mAb (Padlan, Mol. Immunol., 28:489-498, 1991). Therefore, framework residues differing between murine anti-CD40 and the human templates were analyzed and based on solvent exposure were predicted to be buried or located on the surface of the antibody (Padlan, (1991)). Solvent-exposed framework residues distal to the CDRs were not expected to contribute to antigen binding significantly and thus, with the exception of two H chain residues all were changed to the corresponding human amino acid to decrease potential immunogenicity. H chain residues 28 and 46 were predicted to be solvent exposed. However, H28 is located within the HCDR1 region as defined by Chothia & Lesk (1987) and potentially interacts with the antigen. In addition, the lysine at H46 in the murine mAb is somewhat unusual and significantly different from the glutamic acid of the human template. Therefore, the murine and human residues at H28 and H46 were expressed in the combinatorial library (FIG. 1, asterisks).

The remaining differing framework residues, all predicted to be mostly buried within the antibody, were evaluated for: (1) proximity to CDRs, (2) potential to contact the opposite domain in the VK-VH interface, (3) relatedness of the differing amino acids, and (4) predicted importance in modulating CDR activity as defined by Studnicka et. al., Protein Eng., 7:805-814, 1994). The majority of L chain framework differences in buried residues were related amino acids at positions considered not likely to be directly involved in the conformation of the CDR. However, L49 is located adjacent to LCDR2, potentially contacts the VH domain, is unrelated to the human residue, and may be involved in determining the conformation of LCDR2. For these reasons, the murine and human amino acids at L49 were both expressed in the combinatorial framework library (FIG. 1, asterisk).

Analysis of the murine H chain sequence and the human template was performed. Residue H9 is a proline in the murine mAb while the human template contains an unrelated serine residue. Position H9 can also play a role in modulating the conformation of the CDR and thus, was selected as a combinatorial library site (FIG. 1, asterisks). The remaining buried framework residues that differed between murine anti-CD40 and the H chain template were at framework positions 38, 39, 48, and 91. Murine anti-CD40 mAb contained glutamine and glutamic acid at H38 and H39, respectively, while the human template contained arginine and glutamine. Residue H38 is in proximity to the HCDR1, the glutamine-arginine change is non-conserved, and expression of glutamine at this site in murine Abs is somewhat unusual. Similarly, glutamic acid-glutamine is a non-conservative difference for buried amino acids, H39 is a potential VK contact residue, and glutamic acid is somewhat unusual in murine mAbs. Residue H48 is in close proximity to HCDR2 and H91 is predicted to be a high risk site (Studnicka et. al., (1994); Harris & Bajorath, Prot. Sci., 4:306-310, 1995) that potentially contacts the VK domain. Thus, both murine and human residues were expressed at H38, 39, 48, and 91 (FIG. 1, asterisks).

The combinatorial framework library (Hu I) was synthesized, with modifications, by the same method used to construct the chimeric anti-CD40. Overlapping oligonucleotides encoding the framework regions of the H and L variable domains of the human template and the murine anti-CD40 CDRs as defined by Kabat et. al. (1977, 1991) were synthesized. Among these, degenerate oligonucleotides encoding both the murine and the human amino acids at seven VH and one VK framework position as selected above were synthesized (FIG. 1 residues marked with asterisk). All of these sites were characterized by synthesizing a combinatorial library that expressed all possible combinations of the murine and human amino acids found at these residues. The total diversity of this library, termed Hu I, was 28 or 256 variants (Table I).

The Hu I combinatorial library was first screened by an ELISA that permits the rapid assessment of the relative affinities of the variants (Watkins et. al., Anal. Biochem. 253:37-45, 1997). Briefly, microtiter plates were coated with 5 μg/ml goat anti-human kappa (Southern Biotechnology) and blocked with 3% BSA in PBS. Certain Fabs were cloned into an expression vector under the control of the arabinose-regulated BAD promoter. In addition, a six-histidine tag was fused to the carboxyl-terminus of the H chain to permit purification with nickel-chelating resins. Purified Fab was quantitated as described (Watkins et. al., 1997). Next, 50 μl Fab from the Escherichia coli culture supernatant or from the cell lysate, was incubated with the plate 1 h at 25° C., the plate was washed three times with PBS containing 0.1 % Tween 20, and incubated with 0.1 μg/ml CD40-Ig in PBS containing 1% BSA for 2 h at 25° C. The plate was washed three times with PBS containing 0.1% Tween 20 and goat anti-mouse IgG2b-alkaline phosphatase conjugate diluted 3000-fold in PBS containing 1% BSA was added for 1 h at 25° C. The plate was washed three times with PBS containing 0.1% Tween 20 and was developed as described (Watkins et. al., (1997)).

Although variants that bind the target antigen with affinities comparable to, or better than the chimeric Fab were identified, the majority of Hu I clones screened were less active than the chimeric anti-CD40 Fab. Approximately 6% of randomly selected Hu I variants displayed binding activities comparable to the chimeric Fab (data not shown). The identification of multiple Hu I variants with activity comparable to the chimeric CD40 is consistent with the interpretation that the most critical framework residues were included in the combinatorial library.

The kinetic constants for the interaction between CD40 and the anti-CD40 variants were determined by surface plasmon resonance (BIAcore). CD40-Ig fusion protein was immobilized to a (1-ethyl-3-[3-dimethylaminopropyl]-carbodiimide hydrochloride) and N-hydroxysuccinimide-activated sensor chip CM5 by injecting 8 μl of 10 μg/ml CD40-Ig in 10 mM sodium acetate, pH 4. CD40-Ig was immobilized at a low density (˜150 RU) to prevent rebinding of Fabs during the dissociation phase. To obtain association rate constants (kon), the binding rate at six different Fab concentrations ranging from 25-600 nM in PBS was determined at a flow rate of 20 μl/min. Dissociation rate constants (koff) were the average of six measurements obtained by analyzing the dissociation phase. Sensorgrams were analyzed with the BIAevaluation 3.0 program. Kd was calculated from Kd=koff/kon. Residual Fab was removed after each measurement by prolonged dissociation.

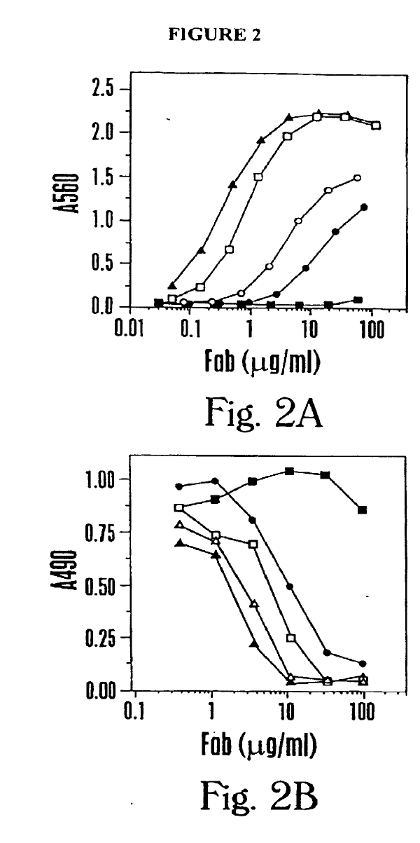

FIG. 2A shows bacterially-expressed chimeric anti-CD40 Fab and certain variants from each of the libraries were titrated on immobilized antigen. Chimeric (filled circles), Hu I-19C11 (open circles), Hu II-CW43 (open squares), Hu III-2B8 (filled triangles), and an irrelevant (filled squares) Fab were released from the periplasmic space of 15 ml bacterial cultures and serial dilutions were incubated with CD40-Ig antigen immobilized on microtiter plates. See below for description of HuII and HuIII libraries. Antibody binding was quantitated as described above. These measurements confirm the identification of multiple variants with enhanced affinity. For example, clone 19C11 binds the CD40 receptor with higher affinity than the chimeric Fab, as demonstrated by the shift in the titration profile (compare open circles with filled circles). DNA sequencing of 34 of the most active clones led to the identification of 24 unique framework combinations, each containing 2-6 murine framework residues (data not shown).

LCDR3 and HCDR3 contact antigen directly, interact with the other CDRs, and often affect the affinity and specificity of antibodies significantly (Wilson & Stanfield, Curr. Opin. Struct. Biol., 3:113-18, 1993); Padlan, (1994)). In addition, the conformation of LCDR3 and HCDR3 are determined in part by certain framework residues. Therefore, to identify the most active antibody it could be beneficial to construct combinatorial libraries that optimize the third CDR of the H and L chains in conjunction with selecting the most active framework.

Codon-based mutagenesis (Glaser et. al., J. Immunol., 149:3903-13, 1992) was used to synthesize oligonucleotides that introduce mutations at every position in HCDR3, one at a time, resulting in the expression of all 20 amino acids at each CDR residue from Ser95-Tyr102 (FIG. 1, underlined). Briefly, for library construction, the overlapping oligonucleotides encoding the framework library and non-library murine CDR were combined with 25 pmol of the oligonucleotides encoding mutated HCDR3. The pool of oligonucleotides encoding the HCDR3 library was mixed with the overlapping oligonucleotides encoding the combinatorial framework and other CDRs to generate a framework/HCDR3 library. The diversity of this library, termed Hu II, was 1.1×10⁵ (Table I).

The CDR residues selected for mutagenesis of LCDR3 were Gln89-Thr97 (FIG. 1, underlined). Oligonucleotides encoding LCDR3 were designed to mutate a single CDR residue in each clone as described above for HCDR3. Oligonucleotides encoding the LCDR3, HCDR3, and the combinatorial framework were used to create a framework/HCDR3/LCDR3 library, termed Hu III. The large number of framework/CDR3 combinations resulted in a library with a complexity of 3.1×10⁷ (Table I).

TABLE II
Summary of phage-expressed anti-CD40 antibody libraries.

| Library | Library Positions | Size* | Screened† |
|---|---|---|---|
| Hu I | framework | 256 | 2.4 × 10³ |
| Hu II | framework, HCDR3 | 1.1 × 10⁵ | 2.0 × 10⁶ |
| Hu III | framework, HCDR3, LCDR3 | 3.1 × 10⁷ | 5.5 × 10⁵ |

*Number of unique clones based on DNA sequence. Thirty-two codons are used to encode all 20 amino acids at each CDR position.

An additional library (Hu IV) was synthesized to further optimize the best variant (clone F4) identified from the Hu III library. Oligonucleotides encoding LCDR3, designed to mutate a single CDR residue in each clone, were synthesized by introducing NN(G/T) at each position (Glaser et. al., (1992)) and were annealed to uridinylated F4 template (Kunkel, (1985)) which already contained a 96R W mutation in LCDR3.

Combining mutations in LCDR3 and/or HCDR3 with the framework library increased the potential diversity of humanized anti-CD40 variants from 256 to greater than 10⁷ To screen these larger libraries more efficiently a modified plaque lift assay, termed capture lift, was used (Watkins et. al., (1997)). Briefly, nitrocellulose filters (82-mm) were coated with goat anti-human kappa, blocked with 1% BSA, and were applied to an agar plate containing the phage-infected bacterial lawn. In the initial screen, phage were plated at 105 phage/100-mm plate. After the capture of phage-expressed anti-CD40 variant Fabs, the filters were incubated 3 h at 25° C. with 5 ng/ml CD40-Ig in PBS containing 1% BSA. The filters were rinsed four times with PBS containing 0.1% Tween 20 and were incubated with goat anti-mouse IgG2b-alkaline phosphatase conjugate (Southern Biotechnology) diluted 3000-fold in PBS containing 1% BSA for 1 h at 25° C. The filters were washed four times with PBS containing 0.1% Tween 20 and were developed as described (Watkins et. al., Anal. Biochem., 256:169-177, 1998). To isolate individual clones, positive plaques from the initial screen were picked, replated at lower density (<10³ phage/100-mm plate), and were screened by the same approach. Because the filters were probed with antigen at a concentration substantially below the Kd of the Fab only variants displaying enhanced affinity were detectable. Multiple clones displaying higher affinities were identified following the screening of >10⁶ variants from Hu II and >105 variants from the Hu III library using 82-mm filters containing 10⁵ variants per filter (Table I). Titration of the variants on immobilized CD40-Ig verified that multiple clones displayed affinities greater than the chimeric and humanized Fab (FIG. 2A, compare open squares, filled triangles with circles).

The framework/CDR mutations that conferred enhanced affinity were identified by DNA sequencing. Single-stranded DNA was isolated and the H and L chain variable region genes were sequenced by the fluorescent dideoxynucleotide termination method (Perkin-Elmer, Foster City, Calif.). Unique variable region sequences were identified in 10/13 Hu II variants and 4/5 Hu III variants. Both the Hu II and Hu III variants contained 1-5 murine framework residues and 0-2 CDR3 mutations. Representative examples are summarized in Table II. The affinities of bacterially-expressed chimeric Fab and certain variants from each of the libraries were characterized more thoroughly using surface plasmon resonance measurements to determine the association and dissociation rates of purified Fab with immobilized CD40-Ig as described above.

Chimeric anti-CD40 had a dissociation constant Kd=48.3 nM and, consistent with the screening results, the variants all displayed higher affinities with Kd ranging from 0.24 nM to 10.5 nM (Table II). Further optimization of LCDR3 of Hu III clone F4 resulted in the identification of a higher affinity (Kd=0.1 nM) clone, L3.17, which contained a 94F→Y mutation. The improved affinities of the anti-CD40 variants were predominantly the result of slower dissociation rates. However, the association rates of most variants were also enhanced, increasing by as much as 3-fold (1.2 vs. 3.2×10⁶ M⁻¹ S⁻¹ for chimeric anti-CD40 and clone L3.17, respectively).

TABLE III
Simultaneous optimization of framework and CDR residues.

| Library | Clone | Kd (nM) | Murine | Fr Residues* | CDR Mutations |
|---|---|---|---|---|---|
| | chimeric | 48.3 | (43) | | 0 |
| Hu I | 19C11 | 42.4 | (2) | H28, 48 | 0 |
| | 1H11 | 53.4 | (4) | H9, 28, 91, L49 | 0 |
| | 9A3 | 43.9 | (3) | H9, 28, 91 | 0 |
| Hu II | CW43 | 10.53 | (3) | H9, 28, 91 | HCDR3, 101A→R |
| | Y49K† | 53.4 | (4) | H9, 28, 91, L49 | HCDR3, 101A→R |
| | 2B12 | 4.67 | (5) | H9, 28, 38, 46, 48 | HCDR3, 101A→K |
| Hu III | 2B12 | 4.67 | (5) | H9, 28, 38, 46, 48 | HCDR3, 101A→K |
| | 2B8 | 2.81 | (1) | H28 | HCDR3, 101A→K |
| | | | | | LCDR2, 96R→Y |
| | F4 | 0.24 | (1) | H28 | HCDR3, 101A→K |
| | | | | | LCDR3, 96R→W |
| Hu IV | L3.17 | 0.10 | (1) | H28 | HCDR3, 101A→K |
| | | | | | LCDR3, 94F→Y |
| | | | | | LCDR3, 96R→W |

*The number of murine framework residues that differ from the most homologous human germline sequence based on definition of CDRs of Kabat et. al. (1977, 1991) are indicated in parentheses. Differing murine framework residues retained in the humanized versions are located predominantly on the H chain (H) at the indicated positions. Hu I clone 1H11 and the CW43 derivative, clone Y49K, contain a single differing L chain (L) framework residue at position 49.
†Clone Y49K was created by site-directed mutagenesis of clone CW43. The four clones within the shaded boxed region, 1H11, 9A3, CW43, and Y49K, were characterized to demonstrate the co-operative interaction between L chain framework residue tyr49 (human) and HCDR3 residue arg101.

The variants displaying enhanced affinity were tested for their ability to block the binding of gp39 ligand to the CD40 receptor. Immulon II microtiter plates were coated with 2 μg/ml anti-murine CD8 to capture sgp39 fusion protein which expresses the CD8 domain. The plates were rinsed once with PBS containing 0.05% Tween 20, and were blocked with 3% BSA in PBS. The plate was washed once with PBS containing 0.05% Tween 20 and was incubated with cell culture media containing saturating levels of sgp39 for 2 h at 25° C. Unbound sgp39 was aspirated and the plate was washed two times with PBS containing 0.05% Tween 20. Next, 25 μl of purified variant Fabs diluted serially 3-fold in PBS was added followed by 25 μl of 4 μg/ml CD40-human Ig in PBS. The plates were incubated 2 h at 25° C. and were washed three times with PBS containing 0.05% Tween 20. Bound CD40-human Ig was detected following a 1 h incubation at 25° C. with goat F(ab′)2 anti-human IgG Fc -specific horseradish peroxidase conjugate (Jackson) diluted 10,000-fold in PBS. The plate was washed four times with PBS containing 0.05% Tween 20 and binding was quantitated calorimetrically by incubating with 1 mg/ml o-phenylenediamine dihydrochloride and 0.003% hydrogen peroxide in 50 mM citric acid, 100 mM Na2HPO4, pH 5. The reaction was terminated by the addition of H2SO4 to a final concentration of 0.36 M and the absorbance at 490 nm was determined. FIG. 2B shows purified variants were tested for their ability to inhibit sgp39 binding to CD40-Ig. The ligand for the CD40 receptor, gp39, was captured in a microtiter plate and subsequently, varying amounts of purified chimeric (filled circles), Hu II-CW43 (open squares), Hu III-2B8 (filled triangles), Hu II/III-2B12 (open triangles), and irrelevant (filled squares) Fab were co-incubated with 2 μg/ml CD40-human Ig on the microtiter plate. The variants all inhibited the binding of soluble CD40-Ig fusion protein to immobilized gp39 antigen in a dose-dependent manner that correlated with the affinity of the Fabs. For example, one of the most potent inhibitors of ligand binding to CD40-Ig fusion protein was variant 2B8, which was also one of the variants with the highest affinity for CD40. Variant 2B8 displayed ≈17-fold higher affinity for CD40 than did the chimeric Fab and inhibited ligand binding ≈7-fold more effectively.

Screening of the Hu I library identified two variants that were similar or identical in framework sequence to the Hu II clone CW43 but displayed 5-fold lower affinities (Table II, clones 1H11 and 9A3). Clone 9A3 has an identical framework structure while 1H11 contained the murine lysine framework residue at L chain position 49. Sequence comparisons and site-directed mutagenesis studies (data not shown) suggest that the beneficial arginine residue at HCDR3 position 101 might interact with L chain residue tyr⁴⁹. To test this, L chain residue tyr⁴⁹ of clone CW43 was mutated to the lysine murine framework residue, resulting in a variant with a framework identical to clone 1H11 that also contained the beneficial arg¹⁰¹ residue in HCDR3. The resulting mAb, termed Y49K, displayed 5-fold lower affinity than CW43. Thus, expression of tyrosine at L chain framework residue 49 or expression of arginine at HCDR3 residue 101 alone had no beneficial effect on the mAb affinity, while the concomitant expression of tyrosine and arginine at these sites improved the mAb affinity 5-fold. The non-additive, or dependent nature of the mutations demonstrates that L chain residue tyr⁴⁹ and HCDR3 residue arg¹⁰¹ interact co-operatively to enhance the affinity of the mAb (Schreiber & Fersht, J. Mol. Biol., 248:478-486, 1995). In addition, the co-operative interaction that was observed between tyr⁴⁹ and arg¹⁰¹ was also observed for variants that expressed lysine at HCDR3 position 101 (Table II).

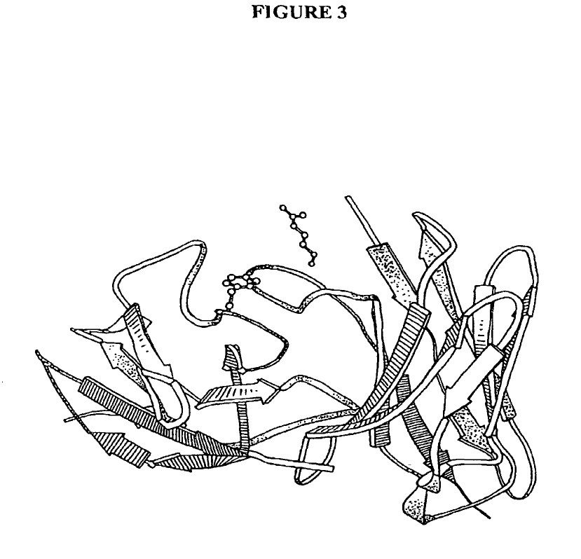

Generally, interacting residues are spatially separated by no more than 7 Å (Schreiber & Fersht, 1995)). FIG. 3 shows molecular modeling of anti-CD40 variant CW43. A top view of the anti-CD40 variant CW43 variable region structure was created by homology modeling to examine the spatial relationship of L chain framework residue Y49 and H chain CDR3 residue RIO. The L chain is on the left and the H chain right with the H chain CDR3 loop depicted in red. The L chain framework residue 49 is in close proximity to the H chain CDR3 loop and is 7Å of the predicted interacting H chain CDR3 R101 residue. Although the interacting amino acids are located on distinct chains of the mAb, the residues are predicted to be within a range (7 Å) to permit co-operative interaction.

### EXAMPLE 2

### Anti-vWF Binding Molecules

This example describes the construction and screen of libraries of anti-human von Willebrand Factor (vWF) Fabs. This example also describes the identification of clones with optimized properties compared to the parental/donor NMC-4 antibody (known to bind human vWF).

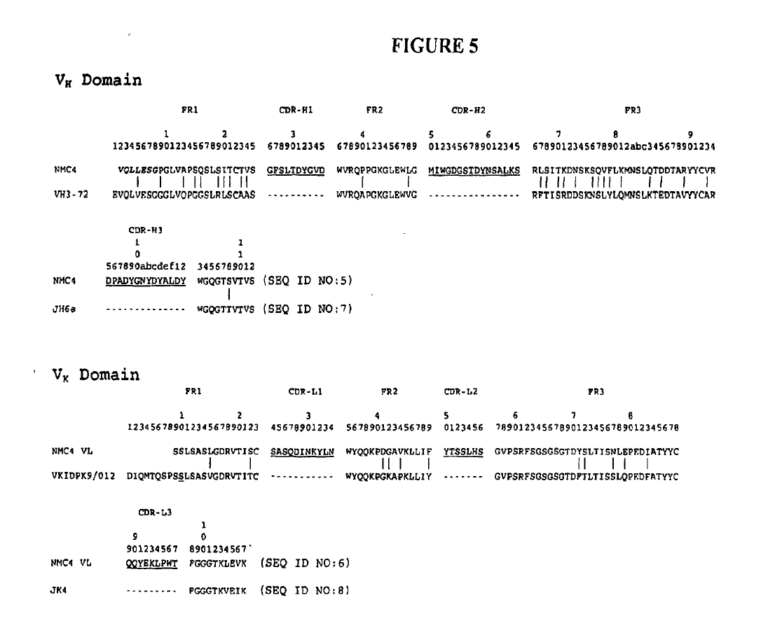

Overlapping oligonucleotides were utilized to generate DNA libraries encoding antibody variants composed of the heavy chain VH3-72 (SEQ ID NO:7) and light chain DPK9/012 (SEQ ID NO:8) human germline framework regions (see FIG. 5) and complementarity-determining regions closely related to those of the NMC-4 antibody (parental/donor antibody). As shown in FIG. 5, the NMC-4 parental/donor antibody is composed of SEQ ID NO:5 (Genbank accession # U90237) and SEQ ID NO:6 (Genbank accession # U90238). The human germline light and heavy chain framework regions utilized were unvaried in this example (i.e. no amino acid residues were changed from these human germline framework sequences). Also in this example, each amino acid position in a number of CDRs were individually randomized to include all amino acids except wild-type. The process generated libraries with a total diversity of 45,486 possible sequences.

The DNA fragments were annealed to uridinylated single stranded phage DNA such that the VL region was inserted between an appropriate signal sequence and the human CL region sequences. Similarly, the heavy chain fragment was designed to insert, in frame, between a signal sequence and the human CH1 region. The phage DNA and the DNA fragments were mixed, heated to 75° C. and cooled to 20° C. over the course of 45 minutes. Double stranded DNA was generated by the addition of T4 DNA polymerase and T4 DNA ligase with an incubation of 5 minutes at 4° C. followed by 90 minutes at 37° C. The reaction was phenol extracted and the double stranded DNA was precipitated by the addition of ethanol. The DNA was resuspended, electroporated into E. coli DH10B cells, XL1 Blue cells were added and the mixture was plated onto agar plates. After 6 hours at 37° C., the phage plaques were counted and eluted into growth media. Phage stocks were generated when the elutions were clarified by centrifugation and sodium azide was added to 0.2%.

Initial screening of the anti-vWF library was performed by plaque lift essentially as described in Watkins, J. D. et al., (1998) Anal. Biochem., 256:169-177, herein incorporated by reference. Briefly, nitrocellulose filters were coated with goat anti-human kappa antibodies and then blocked with 1% BSA. The filters were then placed on agar plates containing plaques from the phage stock described above and incubated for 18 hours at 22° C. Filters were removed from the plates, rinsed with PBS and incubated with various concentrations of biotinylated human vWF. Fab-bound vWF was detected with NeutrAvidin alkaline phosphatase conjugate using a calorimetric substrate. Regions of the agar plate corresponding to the most intense signals were excised, the phages were eluted and amplified and reprobed until discreet positive plaques were isolated. Multiple clones were identified and further characterized by ELISA.

Phage stocks of positive clones from the initial screen were used to infect log phase XL1 Blue which were induced with 1 mM IPTG. After 1 hour at 37° C., 15 ml of infected culture were grown for a further 16 hours at 22° C. Cells were pelleted, washed and the periplasmic contents released by the addition of 640 μof 30 mM Tris pH 8.2, 2 mM EDTA, and 20% sucrose. After 15 minutes at 4° C., the cells were pelleted and the supernatant, containing Fab fragments, was assayed by ELISA. COSTAR #3366 microtiter plates were coated with goat anti-human vWF at 2 μg/ml in carbonate buffer for 16 hours at 4° C. The wells were blocked with 1 % BSA, washed and 0.5 μg/ml human vWF was added to each well for 1 hour at 22° C. After washing, Fab dilutions were added to the wells for 1 hour at 22° C. Goat anti-human kappa alkaline phosphatase was then added for 1 hour at 22° C. Addition of a colorimetric substrate identified clones with the best binding characteristics.

The best clone was the starting point for the generation of individual CDR libraries. Briefly, each CDR was separately deleted by standard mutagenesis methods. Because of its length CDR-H2 was mutagenized as two separate libraries. Uridinylated single stranded DNA templates from each CDR-deleted clone were annealed separately with a pool of oligonucleotides which contained all possible amino acids at each position of the CDR, except wild-type. Double stranded DNA was made and libraries generated as described. Screening was done initially by filter lift, positive clones were assayed by ELISA and the DNA sequence determined. Table 6 summarizes the beneficial mutations identified in the anti-vWF light chain CDRs while the heavy chain summary is shown in Table 7.

TABLE 6
CDR Library Positives - Light Chain

| CDR L1 Library | Clone | | | | | | | | | | |
|---|---|---|---|---|---|---|---|---|---|---|---|
| (11 ammo acids) | 24 | 25 | 26 | 27 | 28 | 29 | 30 | 31 | 32 | 33 | 34 |
| WT | S | A | S | Q | D | I | N | K | Y | L | N |
| L1-6 | | | | | | | | D | | | |

| CDR L2 | Clone | | | | | | |
|---|---|---|---|---|---|---|---|
| (7 amino acids) | 50 | 51 | 52 | 53 | 54 | 55 | 56 |
| WT | Y | T | S | S | L | H | S |
| L2-8 | G | | | | | | |
| L2-1 | N | | | | | | |
| L2-6 | | | | V | | | |
| L2-4 | | | | | | | V |

CDR L3 - all clones have a K93D mutation.

TABLE 7
CDR Library Positives - Heavy Chain

| | |
|---|---|
| CDR H1 Library | Clone |

| (10 amino acids) | 26 | 27 | 28 | 29 | 30 | 31 | 32 | 33 | 34 | 35 |
|---|---|---|---|---|---|---|---|---|---|---|
| WT | G | F | S | L | T | D | Y | G | V | D |
| H1-5 | | | | | G | | | | | |

| | |
|---|---|
| CDR H2A Library | Clone |

| (8 amino acids) | 50 | 51 | 52 | 53 | 54 | 55 | 56 | 57 |
|---|---|---|---|---|---|---|---|---|
| WT | M | I | W | G | D | G | S | T |
| H2a-10 | | | | P | | | | |
| H2a-6 | | | | Q | | | | |
| H2a-1 | | | | | | | | V |

| | |
|---|---|
| CDR H2B Library | Clone |

| (8 amino acids) | 58 | 59 | 60 | 61 | 62 | 63 | 64 | 65 |
|---|---|---|---|---|---|---|---|---|
| WT | D | Y | N | S | A | L | K | S |
| H2b-3 | | I | | | | | | |
| H2b-4 | | | | | | | A | |
| H2b-7 | | | | | | | Q | |
| H2b-1 | | V | | | | | | |

| CDR H3 Library | Clone | | | | | | | | | | | | | |
|---|---|---|---|---|---|---|---|---|---|---|---|---|---|---|
| (14 amino acids) | 95 | 96 | 97 | 98 | 99 | 100 | a | b | c | d | e | f | 101 | 102 |
| WT | D | P | A | D | Y | G | N | Y | D | Y | A | L | D | Y |
| all clones | | | | | | | | | N | | | | | |
| H3-6 | | | | | | | | | | | K | | | |
| H3-9 | | W | | | | | | | | | | | | |

The mutations shown in Tables 6 and 7 were combined into a new library. This combinatorial library was constructed, screened and characterized as described above. Table 8 shows the sequences of clones that had increased binding activity.

TABLE 8
Positive Combinatorial Library Clones

| | LIGHT | | | | | HEAVY | | | | | | | |
|---|---|---|---|---|---|---|---|---|---|---|---|---|---|
| | CDR | | | | | | | | | | | | |
| | L1 | L2 | | | L3 | H1 | H2a | | H2b | | H3 | | |
| | WT | | | | | | | | | | | | |
| | K | Y | S | S | K | T | G | T | Y | K | P | D | A |
| Clone | 31 | 50 | 53 | 56 | 93 | 30 | 53 | 57 | 59 | 64 | 96 | 100c | 100e |
| C1 | D | N | | | D | G | P | | | Q | | N | |
| C4 | D | G | | | D | G | | V | V | Q | | N | |
| C7 | D | N | | | D | G | P | | I | | W | N | |
| C8 | D | N | V | | D | G | P | | V | Q | | N | |
| C9 | D | N | | V | D | G | P | | V | | | D | |
| C4-4 | D | G | | | D | G | | V | V | Q | | D | K |

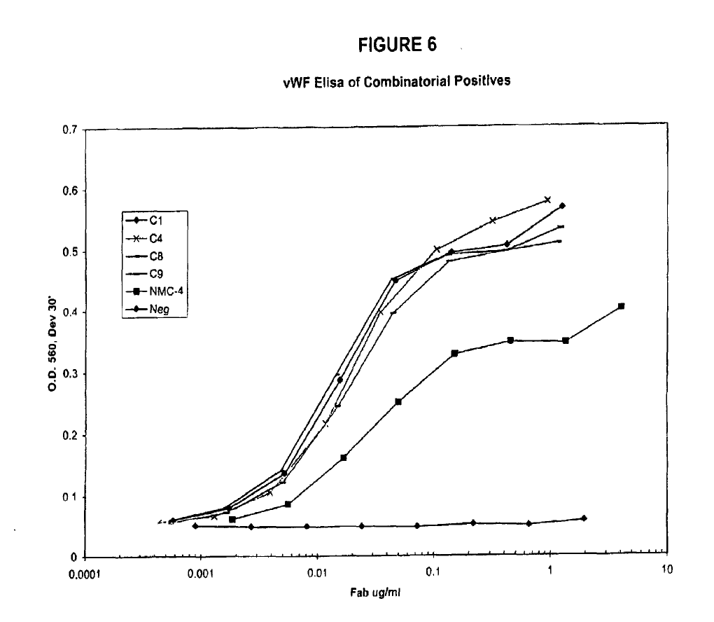

FIG. 6 shows an example of an ELISA in which combinatorial clones were assayed. These results show that the optimized binding activity of many of the clones was 2-3 times greater than the parental/donor NMC-4 antibody.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

1-20. (canceled)

21. A method of expressing a heteromeric variable region having higher antigen binding affinity than a donor heteromeric variable region, wherein said donor heteromeric variable region comprises three light chain donor CDRs and three heavy chain donor CDRs, said method comprising;
a) providing;
i) a first population of oligonucleotides encoding four unvaried human germline light chain framework regions, wherein three of said four unvaried human germline light chain framework regions are from a human kappa light chain gene selected from the group consisting of: A11, A17, A18, A19, A20, A27, A30, L1, L11, L12, L2, L5, L6, L8, O12, O2, and O8;
ii) a second population of oligonucleotides encoding:
A) three light chain CDRs, wherein the three light chain CDRs comprise at least one light chain CDR altered with respect to said light chain donor CDRs
iii) wherein said first population of oligonucleotides and said second population of oligonucleotides overlap to encode a population of light chain variable regions comprising said unvaried human germline light chain framework regions and said light chain CDRs,
iv) a third population of oligonucleotides encoding four unvaried human germline heavy chain framework regions, wherein three of the four unvaried human germline heavy chain framework regions are from a human heavy chain gene selected from the group consisting of: VH2-5, VH2-26, VH2-70, VH3-20, VH3-72, VH-46, VH3-9, VH3-66, VH3-74, VH4-31, VH-18, VH1-69, VH-3-7, VH3-11, VH3-15, VH-3-21, VH3-23, VH3-30, VH3-48, VH4-39, VH4-59, and VH5-51; and
v) a fourth population of oligonucleotides encoding:
A) three heavy chain CDRs, wherein the three heavy chain CDRs comprise at least one heavy chain CDR altered with respect to said heavy chain donor CDRs
vi) wherein said third population of oligonucleotides and said fourth population of oligonucleotides overlap to encode a population of heavy chain variable regions comprising said unvaried human germline heavy chain framework regions and said heavy chain CDRs,
b) mixing said first population of oligonucleotides and said second population of oligonucleotides such that a fifth population of overlapping oligonucleotides is generated, said fifth population encoding said population of light chain variable regions, wherein at least one of said light chain variable regions encoded by said population of fifth oligonucleotides comprises i) an unvaried human germline light chain framework, and ii) at least one altered light chain donor CDR;
c) mixing said third population of oligonucleotides and said fourth population of overlapping oligonucleotides such that a sixth population of oligonucleotides is generated, said sixth population encoding said population of heavy chain variable regions, wherein at least one of said heavy chain variable regions encoded by said population of sixth oligonucleotides comprises; i) an unvaried human germline heavy chain framework, and ii) at least one altered heavy chain donor CDR; and
d) expressing said fifth and sixth populations of oligonucleotides to produce heteromeric variable region binding fragments.

22. The method of claim 21, further comprising step e) identifying at least one heteromeric variable region having higher antigen binding affinity than said donor heteromeric variable region.

23. The method of claim 21, wherein two light chain variable region CDRs are altered compared to said light chain donor CDRs.

24. The method of claim 21, wherein three light chain variable region CDRs are altered compared to said light chain donor CDRs.

25. The method of claim 21, wherein two heavy chain variable region CDRs are altered compared to said heavy chain donor CDRs.

26. The method of claim 21, wherein three heavy chain variable region CDRs are altered compared to said heavy chain donor CDRs.

27. The method of claim 21, wherein said expressing is co-expressing.

28. The method of claim 22, wherein said higher antigen binding affinity is at least 2-fold higher than the affinity of said donor heteromeric variable region.

29. The method of claim 22, wherein said higher antigen binding affinity is at least 3-fold higher than the affinity of said donor heteromeric variable region.


SEQUENCE LISTING (50 sequences)

SEQ ID NO: 1 - protein, 91 aa, mus species
DIVLTQSPATLSVTPGLHWYQQKSHESPRLLIKYASHSISGIPSRFSGSGSGSDFTLSINSVEPEDVGIYYCQHGHSFPRTFGGGTKLEIK

SEQ ID NO: 2 - protein, 107 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 3 - protein, 122 aa, mus species [sequence omitted]

SEQ ID NO: 4 - protein, 112 aa, homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARYFDYWGQGTLVTVS

SEQ ID NO: 5 - protein, 120 aa, mus species
VQLLESGPGLVAPSQSLSITCTVSGFSLTDYGVDWVRQPPGKGLEWLGMIWGDGSTDYNSALKSRLSITKDNSKSQVFLKMNSLQTDDTARYYCVRDPADYGNYDYALDYWGQGTSVTVS

SEQ ID NO: 6 - protein, 96 aa, mus species
SSLSASLGDRVTISCSASQDINKYLNWYQQKPDGAVKLLIFYTSSLHSGVPSRFSGSGSGTDYSLTISNLEPEDIATYYCQQYEKLPWTFGGGTKL

SEQ ID NO: 7 - protein, 81 aa, homo sapiens
EVQLVESGGGLVQPGGSLRLSCAASWVRQAPGKGLEWVGRFTISRDDSKNSLYLQMNSLKTEDTAVYYCARWGQGTTVTVS

SEQ ID NO: 8 - protein, 80 aa, homo sapiens
DIQMTQSPSSLSASVGDRVTITCWYQQKPGKAPKLLIYGVPSRFSGSGSGTDFTLTISSLQPEDFATYYCFGGGTKVEIK

SEQ ID NO: 9 - protein, 11 aa, artificial sequence
  Synthetic Construct
SASQDINDYLN

SEQ ID NO: 10 - protein, 33 aa, artificial sequence
  Synthetic Construct
AGTGCAAGTCAGGACATTAACGACTATTTAAAC

SEQ ID NO: 11 - protein, 7 aa, artificial sequence
  Synthetic Construct
GTSSLHS

SEQ ID NO: 12 - protein, 21 aa, artificial sequence
  Synthetic Construct
GGCACATCAAGTTTACACTCA

SEQ ID NO: 13 - protein, 7 aa, artificial sequence
  Synthetic Construct
NTSSLHS

SEQ ID NO: 14 - protein, 21 aa, artificial sequence
  Synthetic Construct
AACACATCAAGTTTACACTCA

SEQ ID NO: 15 - protein, 7 aa, artificial sequence
  Synthetic Construct
YTSVLHS

SEQ ID NO: 16 - protein, 21 aa, artificial sequence
  Synthetic Construct
TACACATCAGTTTTACACTCA

SEQ ID NO: 17 - protein, 7 aa, artificial sequence
  Synthetic Construct
NTSVLHS

SEQ ID NO: 18 - protein, 21 aa, artificial sequence
  Synthetic Construct
AACACATCAGTTTTACACTCA

SEQ ID NO: 19 - protein, 7 aa, artificial sequence
  Synthetic Construct
YTSSLHV

SEQ ID NO: 20 - protein, 21 aa, artificial sequence
  Synthetic Construct
TACACATCAAGTTTACACGTG

SEQ ID NO: 21 - protein, 7 aa, artificial sequence
  Synthetic Construct
NTSSLHV

SEQ ID NO: 22 - protein, 21 aa, artificial sequence
  Synthetic Construct
AACACATCAAGTTTACACGTA

SEQ ID NO: 23 - protein, 9 aa, artificial sequence
  Synthetic Construct
QQYEDLPWT

SEQ ID NO: 24 - protein, 27 aa, artificial sequence
  Synthetic Construct
CAGCAGTATGAAGATCTTCCGTGGACG

SEQ ID NO: 25 - protein, 10 aa, artificial sequence
  Synthetic Construct
GFSLGDYGVD

SEQ ID NO: 26 - protein, 30 aa, artificial sequence
  Synthetic Construct
GGATTCTCATTAGGCGACTATGGTGTAGAC

SEQ ID NO: 27 - protein, 8 aa, artificial sequence
  Synthetic Construct
MIWPDGST

SEQ ID NO: 28 - protein, 24 aa, artificial sequence
  Synthetic Construct
ATGATATGGCCGGATGGAAGCACA

SEQ ID NO: 29 - protein, 8 aa, artificial sequence
  Synthetic Construct
MIWQDGST

SEQ ID NO: 30 - protein, 24 aa, artificial sequence
  Synthetic Construct
ATGATATGGCAGGATGGAAGCACA

SEQ ID NO: 31 - protein, 8 aa, artificial sequence
  Synthetic Construct
MIWGDGSV

SEQ ID NO: 32 - protein, 24 aa, artificial sequence
  Synthetic Construct
ATGATATGGGGTGATGGAAGCGTA

SEQ ID NO: 33 - protein, 8 aa, artificial sequence
  Synthetic Construct
DINSALKS

SEQ ID NO: 34 - protein, 24 aa, artificial sequence
  Synthetic Construct
GACATTAATTCAGCTCTCAAGTCC

SEQ ID NO: 35 - protein, 8 aa, artificial sequence
  Synthetic Construct
DYNSALAS

SEQ ID NO: 36 - protein, 24 aa, artificial sequence
  Synthetic Construct
GACTATAATTCAGCTCTCGCATCC

SEQ ID NO: 37 - protein, 8 aa, artificial sequence
  Synthetic Construct
DYNSALQS

SEQ ID NO: 38 - protein, 24 aa, artificial sequence
  Synthetic Construct
GACTATAATTCAGCTCTCCAATCC

SEQ ID NO: 39 - protein, 8 aa, artificial sequence
  Synthetic Construct
DVNSALQS

SEQ ID NO: 40 - protein, 24 aa, artificial sequence
  Synthetic Construct
GACGTTAATTCAGCTCTCCAGTCC

SEQ ID NO: 41 - protein, 8 aa, artificial sequence
  Synthetic Construct
DVNSALKS

SEQ ID NO: 42 - protein, 24 aa, artificial sequence
  Synthetic Construct
GACGTTAATTCAGCTCTCAAGTCC

SEQ ID NO: 43 - protein, 14 aa, artificial sequence
  Synthetic Construct
DPADYGNYNYALDY

SEQ ID NO: 44 - protein, 42 aa, artificial sequence
  Synthetic Construct
GACCCAGCCGACTATGGTAACTACAATTATGCTTTGGACTAC

SEQ ID NO: 45 - protein, 14 aa, artificial sequence
  Synthetic Construct
DWADYGNYNYALDY

SEQ ID NO: 46 - protein, 42 aa, artificial sequence
  Synthetic Construct
GACTGGGCCGACTATGGTAACTACAATTATGCTTTGGACTAC

SEQ ID NO: 47 - protein, 14 aa, artificial sequence
  Synthetic Construct
DPADYGNYDYKLDY

SEQ ID NO: 48 - protein, 42 aa, artificial sequence
  Synthetic Construct
GACCCAGCCGACTATGGTAACTACGATTATAAATTGGACTAC

SEQ ID NO: 49 - protein, 14 aa, artificial sequence
  Synthetic Construct
DWADYGNYDYALDY

SEQ ID NO: 50 - protein, 42 aa, artificial sequence
  Synthetic Construct
GACTGGGCCGACTATGGTAACTACGACTATGCTTTGGACTAC